(19) United States
(12) Patent Application Publication
(10) Pub. No.: US 2023/0123815 A1
(43) Pub. Date: Apr. 20, 2023

(54) STABILITY SCORING OF INDIVIDUALS UTILIZING INERTIAL SENSOR DEVICE

(71) Applicant: Proyectos Ingeniería SAS, Montevideo (UY)

(72) Inventors: Hamlet Suárez, Montevideo (UY); Enrique Ferreira, Montevideo (UY); Mariana del Castillo, Montevideo (UY); Francisco de Izaguirre, Montevideo (UY)

(21) Appl. No.: 17/504,939

(22) Filed: Oct. 19, 2021

(51) Int. Cl. G16H 50/20; G16H 50/30; A61B 5/11; A61B 5/00

(52) U.S. Cl. CPC G16H 50/20; G16H 50/30; A61B 5/112; A61B 5/7267

(57) ABSTRACT

Examples herein include a computer-implemented method, a system, and a computer program product, a processor(s) obtains a data sample (data signals and a time vector) from a sensor device proximate to an individual. The processor(s) segments the data sample into segments based on each of the segments into time slices of a pre-defined length of the time period represented by the time vector. The processor(s) determines a physical activity of the physical activities of the individual performed during each time slice. The processor(s) groups times slices by common physical activities. The processor(s) applies a change of basis transformation on data signals of each group to generate data matrices. The processor(s) classify the data matrices into one or more pre-defined stability categories. The processor(s) generate a stability score representing stability of individual when performing the common physical activities.

### FIELD OF INVENTION

The invention relates generally to systems and methods for diagnosing or simulating medical conditions or mining medical data. More specifically, the invention relates to systems and methods for determining health indices for health risk assessment, such as a user stability score or a fall risk score.

### BACKGROUND OF INVENTION

Falling is an everyday risk to individuals and can pose a serious health risk to certain individuals. For example, individuals more prone to falling include the elderly, those with certain health conditions, etc. Although there are factors associated with the risk of falling, there are hurdles to obtaining a comprehensive and accurate assessment. It is important to detect and record the fall, but it is also important to monitor an individual's behavior on a daily basis to assess the risk of falling over time and to predict falling such that preventive measures can be taken to minimize the actual occurrence of falls.

Existing systems can detect when an individual falls and seek assistance, but there are shortcomings to these types of systems, particularly since these systems are reactive measures rather than proactive measures taken to prevent falls from occurring in the first place. Further, existing systems can suffer from inaccuracies due to the use of structured tests and the limited processing of signal data or make the user feel cumbersome due to the use of multiple sensors coupled to the user or in the environment surrounding the user. For example, structured tests can give rise to unnatural behavior because the individual wearing multiple sensors cannot be accustomed to the devices, whereas if only a single sensor device is worn on a daily basis, the user can become accustomed to it and avoid interference from abnormal behavior in calculating the user stability score. Further, accuracy can be improved by transforming data signals to images to use better machine and deep learning algorithms, which result in better, more accurate, classification, and ultimately to a more accurate user stability score.

Thus, there is a need to create a new and useful system and method to assess fall risk. This invention provides such a new and useful system and method.

### SUMMARY OF INVENTION

Shortcomings of the prior art are overcome, and additional advantages are provided through the provision of a method for determining temporal stability of an individual. The method includes, for instance: obtaining, by one or more processors, a data sample from an inertial sensor device, wherein a wearable device comprises the inertial sensor device, wherein the data sample is comprised of data signals and a time vector, wherein the data sample is obtained by the inertial sensor device based on the inertial sensor device monitoring physical activities of a wearer during a time period represented by the time vector; segmenting, by the one or more processors, the data sample into segments, wherein each segment comprises data from a time slice of the time period represented by the time vector; determining, by the one or more processors, a physical activity of the physical activities of the wearer performed during each time slice; grouping, by the one or more processors, into at least one group, times slices in which the wearer performed a common physical activity of the physical activities; applying, by the one or more processors, a change of basis transformation on data signals comprising each group of the at least one group, to generate one or more data matrices; classifying, by the one or more processors, the one or more data matrices generated from each group of the at least one group into one or more pre-defined stability categories; and generating, by the one or more processors, a stability score representing stability of the wearer when performing the common physical activity for each group of the at least one group, based on analyzing the one or more data matrices and weighting the classifications into the one or more pre-defined stability categories.

Shortcomings of the prior art are overcome, and additional advantages are provided through the provision of a system for determining temporal stability of an individual. The system includes a memory, one or more processors in communication with the memory, a sensor device in communication with the one or more processors, and program instructions executable by the one or more processors via the memory to perform a method. The method includes, for instance: transmitting, by the sensor device, to the one or more processors, a data sample from the sensor device, wherein the data sample is comprised of data signals and a time vector, wherein the data sample is obtained by the sensor device based on the sensor device monitoring physical activities of an individual during a time period represented by the time vector; segmenting, by the one or more processors, the data sample into segments, wherein each segment comprises data from a time slice of the time period represented by the time vector; determining, by the one or more processors, a physical activity of the physical activities of the wearer performed during each time slice; grouping, by the one or more processors, into at least one group, times slices in which the wearer performed a common physical activity of the physical activities; applying, by the one or more processors, a change of basis transformation on data signals comprising each group of the at least one group, to generate one or more data matrices; classifying, by the one or more processors, the one or more data matrices generated from each group of the at least one group into one or more pre-defined stability categories; and generating, by the one or more processors, a stability score representing stability of the wearer when performing the common physical activity for each group of the at least one group, based on analyzing the one or more data matrices and weighting the classifications into the one or more pre-defined stability categories.

Shortcomings of the prior art are overcome, and additional advantages are provided through the provision of a computer program product for determining a user stability score. The computer program product comprises a storage medium readable by a one or more processors and storing instructions for execution by the one or more processors for performing a method. The method includes, for instance: obtaining, by one or more processors, a data sample from an inertial sensor device, wherein a wearable device comprises the inertial sensor device, wherein the data sample is comprised of data signals and a time vector, wherein the data sample is obtained by the inertial sensor device based on the inertial sensor device monitoring physical activities of a wearer during a time period represented by the time vector; segmenting, by the one or more processors, the data sample into segments, wherein each segment comprises data from a time slice of the time period represented by the time vector; determining, by the one or more processors, a physical activity of the physical activities of the wearer performed during each time slice; grouping, by the one or more processors, into at least one group, times slices in which the wearer performed a common physical activity of the physical activities; applying, by the one or more processors, a change of basis transformation on data signals comprising each group of the at least one group, to generate one or more data matrices; classifying, by the one or more processors, the one or more data matrices generated from each group of the at least one group into one or more pre-defined stability categories; and generating, by the one or more processors, a stability score representing stability of the wearer when performing the common physical activity for each group of the at least one group, based on analyzing the one or more data matrices and weighting the classifications into the one or more pre-defined stability categories.

Methods, computer program products, and systems relating to one or more aspects are also described and claimed herein. Further, services relating to one or more aspects are also described and can be claimed herein.

Additional features are realized through the techniques described herein. Other embodiments and aspects are described in detail herein and are considered a part of the claimed aspects.

The invention relates generally to systems and methods for diagnosing or simulating medical conditions or mining medical data. More specifically, the invention relates to systems and methods for determining health indices for health risk assessment, such as a user stability score or a fall risk score.

### BRIEF DESCRIPTION OF DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 depicts a diagram of one embodiment of a user stability score system and the method for determining a user stability score;

FIG. 2 depicts a workflow that includes various aspects of some embodiments of the present invention;

FIG. 3 depicts a diagram of one embodiment of a user stability score system and the method for determining a user stability score;

FIG. 4 depicts a workflow that includes various aspects of some embodiments of the present invention;

FIG. 5 depicts a diagram of one embodiment of a user stability score system and the method for determining a user stability score;

FIG. 6 depicts a diagram of an aspect of one embodiment of a user stability score system and the method for determining a user stability score;

FIG. 7 depicts a diagram of an aspect of one embodiment of a user stability score system and the method for determining a user stability score;

FIG. 8 depicts a diagram of one embodiment of a user stability score system and the method for determining a user stability score;

FIG. 9 depicts an example of an inertial sensor device located in a wearable element that can be utilized in embodiments of the present invention;

FIG. 10 depicts a workflow that includes various aspects of some embodiments of the present invention;

FIGS. 11-16 depict examples of scalograms produced from wavelet transformations of data matrices in accordance with embodiments of the present invention;

FIGS. 17-18 depict histograms that illustrates various aspects of some embodiments of the present invention;

FIG. 19 depicts an example of an estimated distribution as determined by the program code in an embodiment of the present invention;

FIG. 20 depicts a computer system configured to perform an aspect of an embodiment of the present invention; and

FIG. 21 depicts a computer program product incorporating one or more aspects of the present invention.

### DETAILED DESCRIPTION

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention. As understood by one of skill in the art, the accompanying figures are provided for ease of understanding and illustrate aspects of certain embodiments of the present invention. The invention is not limited to the embodiments depicted in the figures.

As understood by one of skill in the art, program code, as referred to throughout this application, includes both software and hardware. For example, program code in certain embodiments of the present invention includes fixed function hardware, while other embodiments utilized a software-based implementation of the functionality described. Certain embodiments combine both types of program code.

Examples described herein relate to systems and methods for determining health indices for health risk assessment, including but not limited to, determining whether a given individual is moving in a manner that is stable and, in some examples, assessing and quantifying that stability by assigned a user stability score and/or a fall risk score. As described in greater detail herein, in embodiments of the present invention, program code executing on one or more processing device obtains, from a sensor device, a data sample. This data sample includes the activity/motion-related data, by a wearer of the sensor device, during a set period of time. Upon obtaining the data, the program code segments the data into data that was collected during pre-determined ranges of time (e.g., segments of a pre-defined length and/or segments un which an individual performed a physical activity a set number of times (e.g., time it takes the individual to walk x number of steps)) and then, the program code identifies the activities the user was performing during each segment and groups the segments where the user was performing the same or a similar activity together.

The program code can utilize various motion-related data to determine the activities in which the user was engaging (e.g., gait-type—walking, running, etc.; passive-type—standing, sitting, etc.). Once the like-activity segments are grouped, the program code determined whether the user is stable during those segments/activities. The program code generate/updates/applies a matrix of values for the user related to the given activity, to each group of activities, and based on the relevant matrix, the program code determined whether when performing each activity, the user is stable or unstable (this can be a binary determination or there can be more than one group). Thus, the program code utilizes the matrices to perform a second classification process in order to group various activities, as performed by the user, as indicating that the user is stable or not stable.

Embodiments of the present invention provide significant advantages over existing methods of evaluating and quantifying stability and/or instability of subjects. For example, in some existing approaches, assessing fall risk is limited based on applying structured/static tests to predict when an individual is most at risk of falling. Additionally, these approaches utilize multiple sensors to monitor both the individual and the individual's surrounding environment. This approach is costly and also, because it is complex, is susceptible to errors from all the various pieces of hardware and software that work together to provide a result. The complexity of the hardware components also potentially limits the environments into which these approached can be implemented. As described herein, various embodiments of the present invention address simplify stability assessments and also, provide more accurate stability assessment by utilizing personalized individual data from daily activities in addition to, in some embodiments, only a single sensor device (e.g., one inertial measurement unit (IMU) sensor), to make stability evaluations.

An advantage of examples herein which enables practical applications of various embodiments of the ability of these examples to predict, in a manner relevant to the individual, instability or stability over time. As will be described herein, embodiments of the present invention characterize stability and instability as personal, time dependent, and activity dependent. For example, the caregivers engaged with a patient who has a neurological disorder (e.g., has suffered a stroke, was diagnosed with Parkinson's disease, etc.) and thus has impaired balance and is being treated in a rehabilitation program, can benefit from various aspects of the invention disclosed herein. Aspects of the examples assess therapeutic responses to show an temporal changes, including improvements and/or impairments, of the postural and gait performance of the individual, when the individual is engaged in daily activities. Additionally, patients who are being monitored may change their mobility pattern during day based on various factors, including but not limited to fatigue, post-prandial hypertension, and/or effects of pharmaceuticals prescribed to the patients. Utilizing aspects of the embodiments of to monitor these individuals enables caregivers to establish preventive strategies that avoid fall risks to these patients during specific daily periods. Also the assessment of the patients over longer periods can show changes in the pattern mobility, related to aging and/or disease evolution. Because embodiments of the present invention are personalized, time sensitive (temporal), and can also be activity-specific, aspects of the present invention produce assessments that are more accurate because they are current and personalized.

In addition to providing improvements over existing fall risk assessment methods, as discussed above, embodiments of the present invention are also inextricably linked to computing and directed to a practical application. Aspects of various embodiments of the present invention are inextricably linked to computing at least because various embodiments include an inertial sensor device, a computing device, a computing unit, and a machine-learning system to assess the risk of an individual falling. As described herein, in some embodiments of the present invention, program code executing on one or more processors determines a risk assessment score based on data collected by an inertial sensor device and analyzed by a computing device, utilizing one or more machine learning algorithms. Thus, the program code continually improves the risk assessment methodology applied based on machine learning. Additionally, the purpose of aspects of embodiments of the present invention is practical. By utilizing aspects of embodiments of the present invention, an individual's risk of falling and thus, of injury, is decreased. Examples described herein assess risk of falling with improved accuracy and predictive capabilities, based on daily activities observed by the device described herein, including a single sensor device, e.g., a single IMU. Additionally, implementation of the method serves a practical purpose because, as aforementioned, aspects of the workflow provide immediate user stability scores to prevent imminent risks of falling.

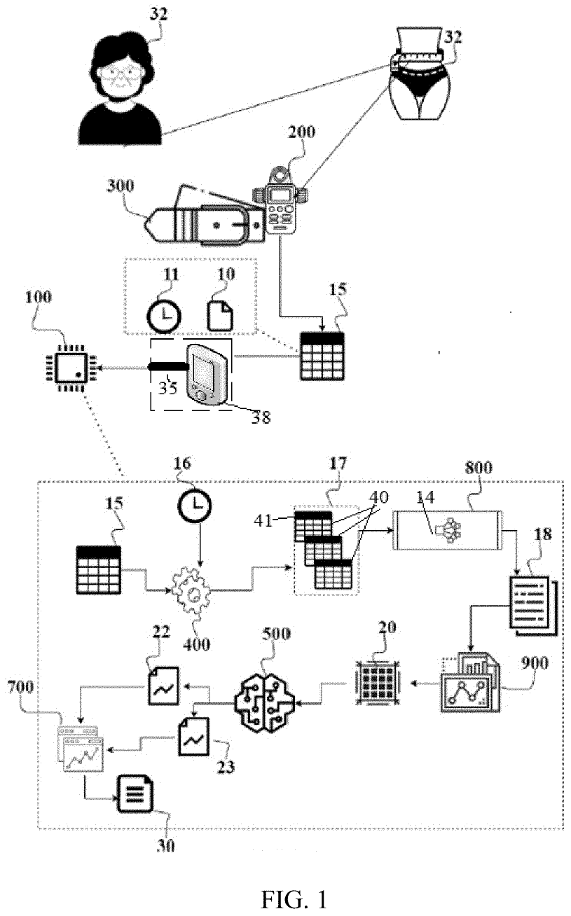

FIG. 1 is a diagram that illustrates various aspects of some embodiments of the present invention. FIG. 1 provides an overview of certain aspects which will be discussed in greater detail herein. As illustrated in FIG. 1, an inertial sensor device 200 is located in a wearable element 300 that is worn by a user 32. The inertial sensor device obtains a data sample 15. The data sample 15 comprises a plurality of data signals 10 and a time vector 11. In some embodiments of the present invention, depending on whether the inertial sensor device 200 is a passive and/or an active sensor, the inertial sensor device 200 can either transmit or the computing unit 100 can obtain, from the inertial sensor device 200, the data sample 15.

Once the program code executing on the computing unit 100 obtains the data sample 15, the program code processes the data sample 15. In some examples, program code executing on one or more processors of the computing unit 100, executes a segmentation process 400 to obtain a plurality of sample segments 17 for a predetermined time range 16. In some examples, the computing unit 100 executes a first classification process 800 to obtain a gait data group 18 from the plurality of sample segments 17. The computing unit 100 can then execute a change of basis transformation process 900, including but not limited to a wavelet transformation process to obtain a plurality of data matrices 20 from the gait data group 18. The data matrices 20 are bidimensional arrays that store the transformation of the data signals 10, which can be interpreted as an image called a scalogram. (The program code can analyze the scalogram to determine whether a user is stable when performing a given activity.) The computing unit 100 can further execute a second classification process 500 to obtain one or more data groups 22, 23, including but not limited to, A-type stability data group 22 (time segments in which the user is evaluated to be performing activities in a manner that is stable) and B-type stability data group 23 (time segments in which the user is evaluated to be performing activities in a manner that is unstable). These groups indicate quantitative or qualitative levels of stability. For example, in some embodiments, there are only two groups and grouping in one group or the other indicates stability or instability of a user who is engaged in a given activity (during the time segments that are grouped together based on the activity being identified). In this binary example, A-type stability data group 22 includes sample segments 17 classified as stable in a particular user activity 40. For example, the program code can classify sample segments 17 is the A-type stability data group 22 that corresponding to time ranges when the user 32 is walking on stairs. Meanwhile, the program code assigns segments to the B-type stability data group 23 that the program code classifies as unstable in a particular user-activity 40. For example, the B-type stability data group 23 can group the data matrices 20 obtained from sample segments 17 corresponding to time ranges when the user 32 is walking on stairs and classify these as unstable. The computing unit 100 can further execute a weighting process 700 to obtain a stability score data 30 from the one or more data groups 22, 23. Each of these aspects is discussed in greater detail herein.

As discussed above, the program code of the computing unit 100 obtains the data sample 15 from the inertial sensor device 200. In different embodiments, the inertial sensor device 200 can transmit the data sample 15 and/or the program code executing on the computing unit 100 can query the inertial sensor device 200 to obtain the data sample 15. The data sample 15 can include a plurality of data signals 10 and a time vector 11. In some examples, the data sample 15 can comprise acceleration signals related to different coordinate axis, e.g., cartesian 2-dimensional D) axis, cartesian 3D axis, polar axis, spherical axis, etc., and rotation rates related to those different coordinate axis. In some examples, the data signals 10 include one or more of six 6 signals provided by the inertial sensor device 200: X-axis acceleration, Y-axis acceleration, Z-axis acceleration, Y-gyroscope angular velocity, Y-gyroscope angular velocity, and Z-gyroscope angular velocity. In some examples, the plurality of data signals 10 are selected from three-axis-low noise acceleration, three-axis wide range acceleration, three-axis-gyroscopic angular rate, and combinations thereof. In some examples, the time vector 11 is an array, vector, or a series that stores and correlates the time variable associated with each data signal in the plurality of data signals 10.

Before the signals in the data sample 15 are provided to the program code of the computing unit 100, and/or after the signals are obtained, the signals can be pre-processed by one or more of the computing unit 100 and the inertial sensor device 200. The signal preprocessing method can include, but is not limited to, techniques such as signal filtering (e.g., limiting the range in frequency), normalization (e.g., restricting the signal to values between 0 and 1, amplification (e.g., modifying the signal amplitude with a function), dynamic time warping (DTW), other techniques known by a person of ordinary skill in the art, and/or combinations thereof. In some examples, the DTW techniques comprise synchronizing two signals.

Referring to FIG. 1, the data sample 15 can be sent to or obtained by the computing unit 100. In this example, program code executing on one or more processors of the computing unit 100 executes the segmentation process 400 to obtain a plurality of sample segments 17 for a predetermined time range 16. In this example, the predetermined time range 16 is the time interval used to split the data sample 15 into sample segments 17. In some examples, the predetermined time range 16 is selected from the range between ˜3 s and ˜5 s. The predetermined time range 16 can be determined by a data-analytics process of a plurality of data samples 15 and related stability score data 30. In some examples, the predetermined time range 16 is configured by a user as it was determined by an expert or operator. In some examples, the program code calculates the predetermined time range 16 or a user configures the time range 16 and it remains static.

As will be discussed in greater detail herein, the length of the predetermined time range 16 affects the functionality of the embodiments. For example, a longer predetermined time range 16 generally means each sample segment 17 comprises more information, and it is possible for a sample segment 17 to contain more than one user activity 40. When the segment includes more than one activity, the program code can classify the sample segment 17 being classified as “unclassified” in the first classification process 800. A shorter predetermined time range 16 generally means each sample segment 17 comprises less information, and it is more likely that a sample segment 17 will contain one user-activity 40, resulting in the sample segment 17. In this example, the program code can more accurately classify this segment utilizing the first classification process 800. In some embodiments of the present invention, the data matrices 20 dimension is related to the sample segments 17 dimension, which is defined by the predetermined time range 16.

As discussed below, as part of a first classification process 800, program code executing on one or more processors of the computing unit 100 classifies various data segments, that represent different periods of time, into activity groups. These activity groups represent the types of activities the user was partaking in during the time segments. The program code determines and/or distinguishes the types activities utilizing the sensor data. The program code classifies the time segments based, at least in part, by the program code applying logic to data collected the sensor during the time range. The data collected during this time range will include data related to activities performed by the user who is wearing the sensor). A user activity 40 can be any activity that the user 32 can perform and which the first classification process 800 is trained to classify. In some examples, the user activities include, but are not limited to, walking, walking on stairs, standing, sitting, and running. A user activity related data group 41 includes all the sample segments 17 that are classified for the same user activity 40. For example, a walking data group includes all sample segments classified as walking.

In some embodiments of the present invention, sample segments 17 have the same or a similar structure as the data sample 15 but span shorter time periods, e.g., predetermined time ranges 16. The segmentation process 400 performed by the program code splits the data sample 15 into segments of equal length, i.e., sample segments 17. In some examples, the program code performed the segmentation process 400 by the program code executing on one or more processors of the computing unit 100 determining the time length of the data sample 15 and calculating how many times the predetermined time range 16 fits into the data sample 15 without overlap, followed by the splitting of the data sample 15 into that many sample segments 17. In some examples, the program code slices the data sample 15 into fixed-time sample segments to obtain sample segments 17.

Referring to FIG. 1, program code executing on one or more processors of the computing unit 100 can further execute a first classification process 800 to obtain a gait data group 18 from the plurality of sample segments 17. (Gait describes a manner in which an individual walks and thus, gait is used herein to signal activities and time periods where a user is in motion.) The program code performs the first classification process to group the sample segments 17 into different classes according to performed user activities 40. The gait data group 18 comprises the sample segments 17 classified as corresponding to a dynamic activity. In some examples, the gait data group 18 comprises all sample segments 17 obtained from the data sample 15. In some examples, the gait data group 18 comprises only the sample segments 17 obtained from the data sample 15 that have a valid value (e.g., that have values within ranges that indicate motion).

The program code in some examples herein performs the first classification process 800, to classify the sample segments 17 based on user activity 40. In some examples, the program code classifies the sample segments into pre-defined groups: a gait activity group, a passive activity group, and an unclassified group. The program code can classify a user activity 40 as a gait activity, which is an activity when the user 32 is moving as opposed to being still. For example, a gait activity includes, but is not limited to, walking, walking on stairs, jogging, running, jumping, dancing, or any other activity performed when the user 32 is moving. In some examples, the program code classifies segments 17 into the gait data group including, but not limited to, sample segments 17 corresponding to time ranges when the user 32 is doing a gait activity such as walking, walking on stairs, etc. The program code can classify segments into the passive activity data group where sample segments 17 correspond to time ranges when the user 32 is not doing a gait activity, such as when the user 32 is doing passive activities such as sitting, standing, etc. The program code can classify segments into the unclassified data group 43 that include sample segments 17 that could not be classified under one user activity 40 through the first classification process 800, such as when the sample segment contains two different user activity 40 groups (e.g., walking at the beginning of the sample segment 17 and standing still at the end of the sample segment 17).

In performing the first classification process 800, the program code determines an activity or type of activity the user was performing in each time segment of data provided by the sensor.

In some examples, the first classification process 800 can comprise artificial intelligence and machine learning processes, such as support vector machines, linear classification processes, k-nearest neighbors, decision trees, gradient boosted trees, random forests, neural networks, fully connected neural networks, logistic regression processes, Fisher's linear discriminant, quadratic classifiers, other similar techniques known by a person of ordinary skill in the art, or combinations thereof.

In some examples, prior to the first classification process 800, a characteristics vector 14 is generated for each sample segment 17, wherein the characteristics vector 14 can include at least one parameter selected from mean, variance, standard deviation, energy, leading frequencies, maximum values, minimum values, correlation values, other metrics or statistical parameters known by a person of ordinary skill in the art, and combinations thereof. In some examples, the first classification process 800 is applied to the characteristics vector 14, which can include but are not limited to a matrix, tensor, equivalents, and/or combinations thereof.

In some examples, the first classification process 800 is a previously trained process. The program code is trained based on the program code obtaining a plurality of data samples 15, splitting the data samples 15 into one or more training data groups, and one or more testing data groups, and the program code obtaining a plurality of performance parameters for the first classification process 800, according to a machine learning process. The machine learning process can include adjusting the first classification process 800 according to the training data group and the testing data group and obtaining hyperparameters (e.g., variable weights, deep limit, number of estimators, learning rate, among others) of the first classification process 800 to increase the accuracy and predictive precision.

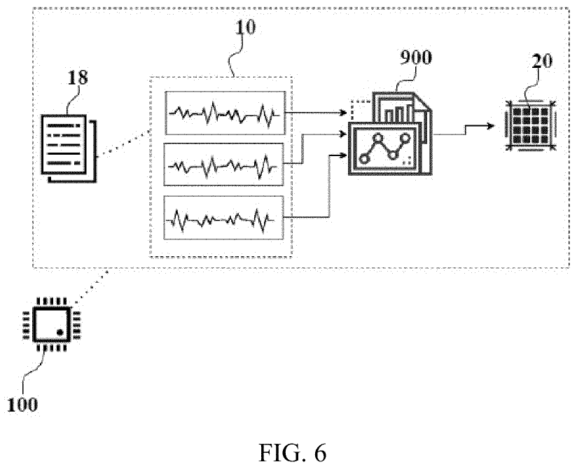

As aforementioned, program code executing on one or more processors of the computing unit 100 can further execute a first classification process 800 to determine which sample segments comprise a gait data group 18 from the plurality of sample segments 17. Having determined which sample segments to include in the gait group, the program code executing on one or more processors of the computing unit 100 can further execute a change of basis transformation process 900, such as a wavelet transformation process, to obtain a plurality of data matrices 20 from the gait data group 18. Specifically, program code in embodiments of the present invention applied a change of basis transformation process 900, such as a wavelet transformation process, to at least a data signal 10. The program code takes as input, to this wavelet transform process 900 at least one data signal 10 from the sample segments 17. The program code generates data matrices 20 that are one or more bidimensional arrays that store the wavelet transform of the data signals 10. In some examples, the program code interprets the data matrices 20 as images, i.e., scalograms. The wavelet transform process 900 is described in greater detail below including in FIG. 6. However, the wavelet-transform process 900 can include methods and models included in libraries (e.g., Pywavelets (SciPy, PyCWT, ObsPy)).

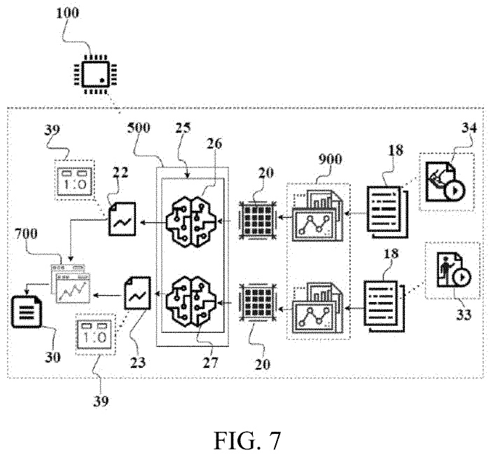

As discussed above and as depicted in FIG. 1, in some embodiments of the present invention, program code executing the computing unit 100 executes a second classification process 500 to obtain data groups 22, 23, for example, A-type stability data group 22 (i.e., a stable group) and B-type stability data group 23 (i.e., an unstable group), from the data matrices 20. The second classification process 500 can comprise classifying the sample segments 17 of a user activity related data group 41 according to stability classes or values that can be categorical or numerical. In some examples, the second classification process 500 classifies the sample segments 17 in the gait data group 18 as stable, unstable, or in intermediate scales of stability. The second classification process 500 is described in greater detail below (see FIG. 7).

Referring to FIG. 1, the computing unit 100 can further execute a weighting process 700 to obtain a stability score data 30 from the data groups 22, 23. The weighting process 700 can comprise employing a mathematical combination that sums the number of sample segments 17 in each data group and multiplying each sum by a fixed parameter to obtain a final outcome. In some examples the weighting process 700 comprises a mathematical- or statistical-based processes, tests, or plausibility functions to extract a mean value or weighted value from a data sample. In some examples, the weighting process 700 can comprise a classification process to classify the sample segments 17 in a data group into certain categorical or numerical levels or values of stability. In some examples, the weighting process 700 can comprise a regression process, e.g., logistic regression, linear regression, logarithmic regression, etc., or a classification process, previously trained with data samples 15 having a known stability score data 30.

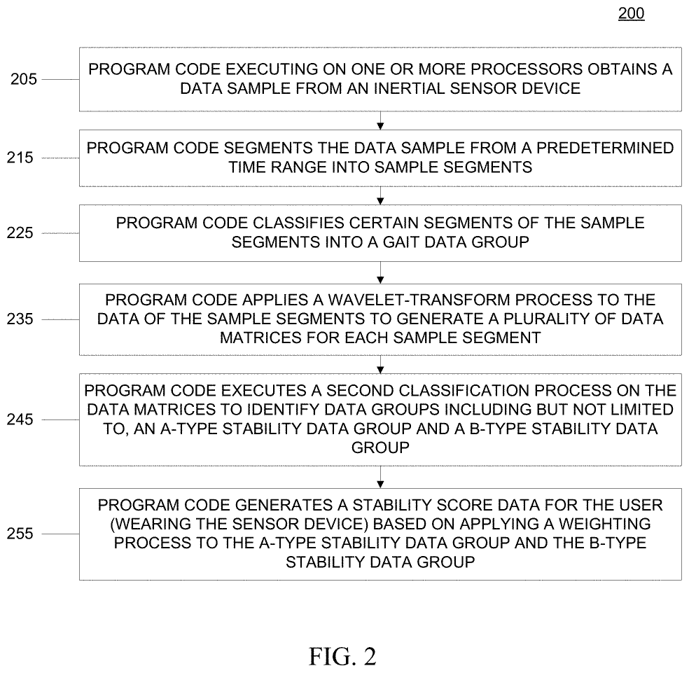

FIG. 2 is a workflow 200 that illustrates various aspects of some embodiments of the present invention. For illustrative purposes only, various aspects in the workflow 200 are illustrated with references to aspects illustrated in other figures herein. As illustrated in FIG. 2, in some embodiments of the present invention, program code executing on one or more processors obtains a data sample 15 from an inertial sensor device 200 (e.g., with one sensor, where the sensor is worn by a user) (205). The program code can be executed on one or more of a computing unit 100 and the inertial sensor device 200, as in, the data sample 15 can be transmitted to the computing unit 100 and/or the computing unit 100 can obtain data from the sensor. In some embodiments, before the program code obtains the data sample 15, the program code (executed on processing resources of the sensor device and/or the computing unit 100) can apply a screening schedule such that the data transmitted and/or received is in accordance with the schedule. The program code can obtain the screening schedule based on entry of the schedule by a user 32 or an operator. In some examples, the operator is a person who supervises the stability score data 30 results, such as a physician, medic, nurse, caregiver, the user 32, or a combination thereof. In some examples, the screening schedule can be a time data group containing hour ranges when it is desired to obtain data samples 15, and the information registered in the time data group can be generated with a user interface of the computing unit 100, the inertial sensor device 200, or an external the computing device 38. Please note that FIG. 1 illustrates the sensor device 200 being communicatively coupled to the computing unit 100 as well as an example where the sensor device 200 is communicatively coupled to the computing device 38, which in turn communicates with the computing unit 100, in this example via an application, which is depicted in this example as an application programming interface (API) 35. The user interface can be used by the operator to enter the hour ranges when it is desired to obtain data samples 15. In some examples, the operator can select hour ranges when it is known that the user 32 is doing more gait activities. In some examples, the screening schedule is preconfigured as a setting for any user 32. Generally speaking, the program code will evaluate data collected by the sensor during specified periods of time.

Returning to FIG. 2, the program code segments the data sample 15 from a predetermined time range 16 into sample segments 17 (215). The program code classifies certain segments of the sample segments 17 into a gait data group 18 (225). The program code applies a wavelet-transform process 900 to the data 10 of the sample segments 17 to generate a plurality of data matrices 20 for each sample segment 17 (235). The program code executes a second classification process 500 on the data matrices 20 to identify data groups (based on grouping data by perceived stability with which a user is performing the activities being performed by the user in the data segments), including but not limited to, an A-type stability data group 22 (e.g., stable) and a B-type stability data group 23 (e.g., unstable) (245). The program code generates a stability score data 30 for the user (wearing the sensor device) based on applying a weighting process to the A-type stability data group 22 and the B-type stability data group 23 (255).

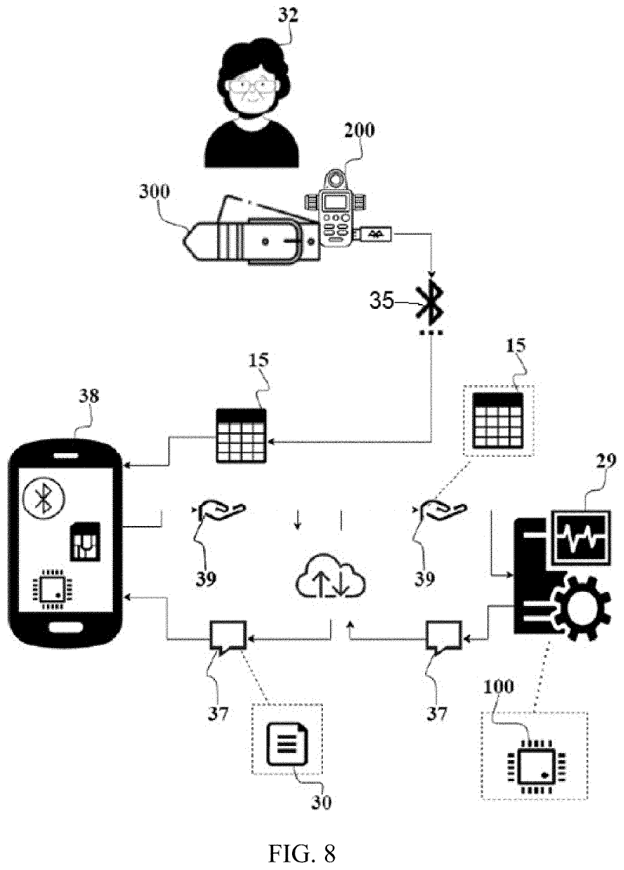

A data sample 15 can include data 10 that do not correspond to normal movements of the user 32. For example, if the wearable element 300 is not worn properly such that the inertial sensor device 200 is not positioned at the lower back of the user 32, as described in below (see FIG. 8). The present method allows for the processing of data samples 15 obtained while the user 32 is performing normal, every-day activities. Although the method may not require structured tests, it is possible for the user 32 to add structured tests to the APP 35.

In some examples, program code executing on one or more processors obtains a data sample 15 from an inertial sensor device 200 (205) by receiving the data sample 15 from an inertial sensor device 200 which is connected to a computing device 38 and the data sample 15 is sent to a computing unit 100 by the computing device 38. In some examples, the computing unit 100 can include one or more servers connected remotely to the computing device 38 by a local computer network, internet, etc. FIG. 8 depicts various aspects of this computing environment in more detail. But returning to FIG. 2, as illustrated by aspects in FIG. 1, the computing device 38 computing unit 100, which in this example is one or more servers, can communicate through an application programming interface (API) 35 or other application, configured to be accessed or executed by the computing device 38. A computer network can be a technical means that allows remote communication between autonomous equipment, such as computing devices 38, and computing units 100, the latter which ca include servers. Non-limiting examples of a communications network are the Internet, WAN, and LAN.

In some embodiments, program code segments the data sample 15 from a predetermined time range 16 into sample segments 17 (215) by obtaining a plurality of sample segments 17 from the data sample 15 by executing a segmentation process 400 with a predetermined time range 16.

In some embodiments, program code classifies certain segments of the sample segments 17 into a gait data group 18 (225) by obtaining user activity related data groups 41, such as a gait data group 18, a passive activity data group, an unclassified data group, etc., by performing a first classification process 800. The program code analyzes the data and classifies sample segments 17, based on the data therein, into predefined user activities such as walking, walking on stairs, standing, sitting, etc. Thus, the program code identifies the user activity 40 that was performed by the user who was wearing the sensor during each segment. In some embodiments, the program code validates the data sample it receives as only certain data can be utilized in evaluating stability (e.g., based on activities being performed by the user during the times in which the data is collected). If the program code obtains data that cannot be utilized to evaluate stability of the user (e.g., a null gait data group 18), the program code can automatically alert various entities, (e.g., the user, the sensor device) such that new data can be collected for evaluation. This validation process 600 include the program code sending an alert if the preliminary validation process does not obtain valid data 13. For example, the alarm can include notifying the user 32 that another data sample 15 should be acquired.

As aforementioned, the program code applies a wavelet-transform process 900 to the data 10 of the sample segments 17 to generate a plurality of data matrices 20 for each sample segment 17 (235). Thus, the program code determines, when evaluating different segments in which a user is performing the same activity (as identified by the program code) when the user is stable and when the user is unstable in performing the activity. As noted above, in some examples, the program code identifies segments in which a user performs a given physical activity as with stable or unstable, separating the segments into an A-type stability data group 22 and a B-type stability data group 23. The program code makes this separation because based on grouping data related to certain specific activities together, the program code can generate data matrices 20 that provide a representation of ranges for behaviors of a given user when that user is performing a given activity. Thus, the program code can identify stability or lack thereof via the matrices. The program code in embodiments of the present invention is self-learning at least because the longer data is collected from the sensor for a given individual and analyzed by the program code, the more detailed and tuned the matrices can become. In some embodiments of the present invention, the program code applies the second classification process 500 to the data matrices 20 (generated from the grouped segment data). The program code groups various segments into an A-type stability data group 22 (stable) or a B-type stability data group (unstable) 23. To make these classifications, the program code compares data related to segments in which a user is engaged in a particular user activity 40. For example, the A-type stability data group 22 can be based on data matrices 20 generated by the program code based on data from sample segments 17 corresponding to time ranges when the user 32 is walking on stairs and are classified as stable. In some examples, the B-type stability data group 23 can group the data matrices 20 corresponding to sample segments 17 classified as unstable in a particular user-activity 40. For example, the B-type stability data group 23 can be based on data matrices 20 generated by the program code based on data from sample segments 17 corresponding to time ranges when the user 32 is walking on stairs and are classified as unstable. In some examples, the program code applies the second classification process 500 to classify the sample segment 17 in two or more stability data groups.

As noted above, the program code applies a wavelet-transform process 900 to the data 10 of the sample segments 17 to generate a plurality of data matrices 20 for each sample segment 17 (235), which can include the program code generating a plurality of data matrices 20 for each sample segment 17 of the user activity related data groups 41. In some examples, the program code generates the data matrices by applying a wavelet transform process 900 to the data 10 of the sample segments 17.

As illustrated in FIG. 2, the program code generates a stability score data 30 for the user (wearing the sensor device) (245). In some examples, the program code generates a stability score data 30 for the user 32 for each user activity 40. In some examples, the program code generates the score(s) by weighing different data groups, e.g., A-type stability data group 22 and B-type stability data group 23. The weighing process 700 credits stability and instability of a user performing various activities.

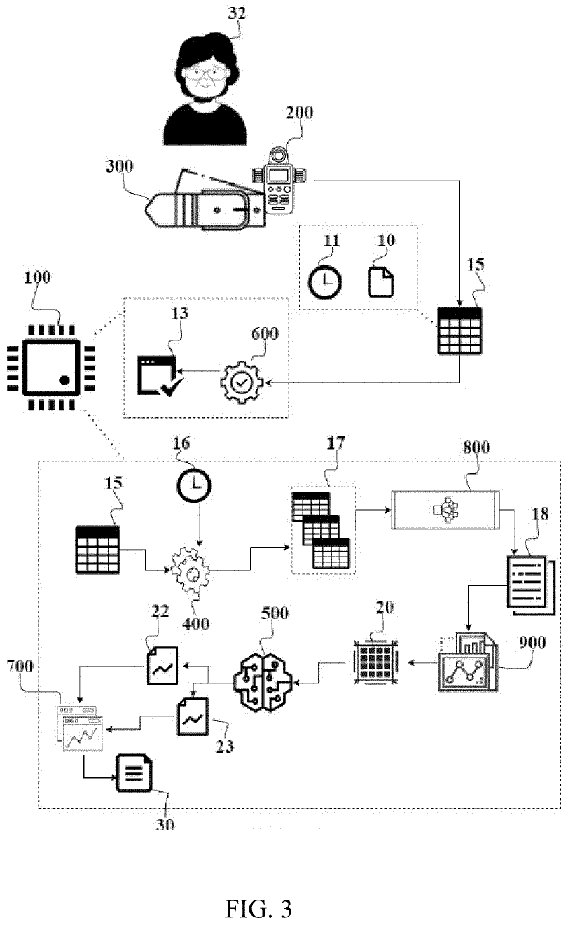

FIG. 3 illustrates various aspects of some embodiments of the present invention where the program code validates data collected from the sensor. As illustrated in FIG. 3, an inertial sensor device 200 is located in a wearable element 300 that is worn by a user 32 and from which the program code obtains a data sample 15. The data sample 15 comprises a plurality of data signals 10 and a time vector 11 (i.e., the data is collected during a set window of time). As discussed with reference to FIG. 2, the program code (executing on one or more processor of the sensor device and/or a computing unit external to the sample device) validates the sample by applying a preliminary validation process 600. In this manner, the program code determines whether the collected data is valid data 13. Once validated, if the data were validated at the sensor, the valid data 13 are transmitted to a computing unit 100. The program code of the computing unit 100 (and/or program code executing on the sensor device) executes a segmentation process 400 to obtain a plurality of sample segments 17 from the valid data 13 for a predetermined time range 16. Thus, the program code segments a data sample from a larger time range into data samples from smaller (pre-determined) time ranges. The program code executing on processing resources of the computing unit 100 can further execute a first classification process 800 to obtain a gait data group 18 from the plurality of sample segments 17. The computing unit 100 can further execute a wavelet transform process 900 to generate a plurality of data matrices 20 from the gait data group 18. The computing unit 100 can further execute a second classification process 500 to classify segments into groups based on evaluated stability (per the matrix) (e.g., A-type stability data group 22 and B-type stability data group 23.) The computing unit 100 can further execute a weighting process 700 to generate a stability score data 30 from the data groups 22, 23.

The preliminary validation process validates a data segment 17 by comparing the acceleration value of an acceleration signal with a predetermined acceleration range. Acceleration data can include information related to acceleration signals contained in the data signals 10 of a sample segment 17. In some examples, the acceleration data can include a mean value or a peak value of an acceleration signal contained in the data signals 10 of the data sample 15. A predetermined acceleration range can be a range or interval of acceleration values that are considered valid. For example, the predetermined acceleration range can be an interval in which the acceleration according to the vertical axis must fall.

In some examples, the preliminary validation process 600 comprises the program code executed conditions that compare if the acceleration value of the acceleration signal falls within the predetermined acceleration range. In some examples, the data sample 15 can include situations that do not represent one user activity 40, in which cases, the preliminary validation process 600 improves the accuracy of the stability score data 30. If the acceleration value of an acceleration signal passes the preliminary validation process 600, then the computing unit 100 can use the data sample 15 to generate a stability score data 30. If the acceleration value of an acceleration signal fails the preliminary validation process 600, and the data sample 15 is not validated, the step of obtaining a data sample 15 can be repeated and a new data sample 15 obtained. In some examples, the predetermined acceleration range is programmed.

In some examples, the acceleration data in the data sample 15 can comprise data signals 10 of movements with irregular acceleration magnitudes, e.g., vertical acceleration signals having a mean value of less than 8 m/s², or more than 10 m/s². In some examples, an admissible mean value for the magnitude of a vertical acceleration signal can be between 8 m/s² and 10 m/s². The preliminary validation process 600 avoids further processing of data samples 15 comprising abnormal movements.

In some examples, the program code executing the preliminary validation process 600 processes information contained in the data sample 15, such as mean values of magnitudes of accelerations in horizontal axes, polar axes or spherical axes, mean values of magnitudes of turning rate-signals, etc. which can be obtained by the inertial sensor device 200. In some examples, the preliminary validation process 600 can comprise one or more predetermined acceleration ranges associated with a coordinate axis.

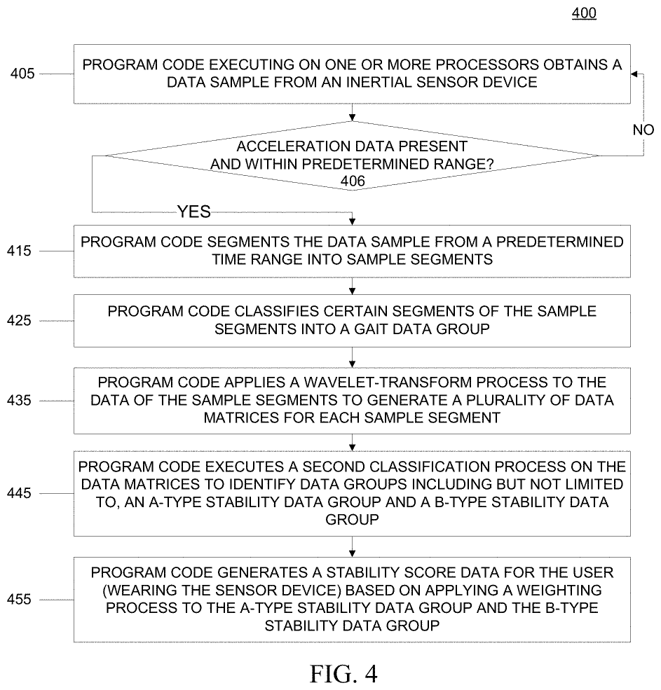

FIG. 4 is a workflow 400 that illustrates various aspects of some embodiments of the present invention. As illustrated in FIG. 4, in one embodiment, the method comprises various aspects also illustrated in FIG. 2. A difference between the workflow of FIG. 4 and the workflow of FIG. 2 is that the workflow 400 of FIG. 4 includes the program code executing a preliminary validation process 600 that takes as input, acceleration data contained in the data sample 15, and a predetermined acceleration range.

Referring to FIG. 4, in some embodiments of the present invention, program code executing on one or more processors obtains a data sample 15 from an inertial sensor device 200 (e.g., with one sensor, where the sensor is worn by a user) (405). The program code, executing on one or more processors of the computing unit and/or on the inertial sensor device 200 itself, validates the data sample 15 to determine if the data sample 15 comprises acceleration data within a predetermined acceleration range (406). Based on determining the acceleration data is within the predetermined acceleration range, the program code continues with additional aspects, as illustrated in both FIG. 2 and FIG. 4. However, should the program code determine that the acceleration data obtained is not valid, for example, that it is not within the predetermined acceleration range, the program code obtains a new data sample and validates the new data sample for the acceleration data presence within the same manner, progressing only once this acceleration data validation is complete.

Returning to FIG. 4, after validating the acceleration data (406), the program code segments the data sample 15 from a predetermined time range 16 into sample segments 17 (415). The program code classifies certain segments of the sample segments 17 into a gait data group 18 (425). The program code applies a wavelet-transform process 900 to the data 10 of the sample segments 17 to generate a plurality of data matrices 20 for each sample segment 17 (435). The program code executes a second classification process 500 on the data matrices 20 to identify data groups (based on grouping data by perceived stability with which a user is performing the activities being performed by the user in the data segments), including but not limited to, an A-type stability data group 22 (e.g., stable) and a B-type stability data group 23 (e.g., unstable) (445). The program code generates a stability score data 30 for the user (wearing the sensor device) based on applying a weighting process 700 to the A-type stability data group 22 and the B-type stability data group 23 (455). The additional examples that are provided in the discussion of FIG. 2 are all examples in the workflow 400 of FIG. 4, provided that there are no inconsistencies.

Returning to FIGS. 1, 3, 5, and 7, the second classification process 500 can include one or more classification processes, each classification process configured to determine whether segments of time in which the user is performing distinct activities indicate that the user is stable or unstable during these times and thus, whether the program code should assign these segments to two or more types of stability data groups (e.g., the A-type stability data group 22, a more stable group, and the B-type stability data group 23, a less stable group). Each classification process is configured to classify a plurality of sample segments in a particular user activity related data group 41.

For example, program code of one of the classification processes of the second classification process 500 is trained to assign the A-type stability data group 22 and the B-type stability data group 23 for walking data group segments. The program code can group A-type stability data group 22 using a data matrices 20 the program code generated from sample segments 17 corresponding to time ranges when the user 32 is walking and are classified as stable. The B-type stability data group 23 can group the data matrices 20 generated by the program code from sample segments 17 corresponding to time ranges when the user 32 is walking and are classified as unstable. The activity can be any predefined user-activity 40.

Similarly, another classification process of the second classification process 500 can be trained to identify the A-type stability data group 22 and the B-type stability data group 23 for walking on stairs data group segments. The A-type stability data group 22 can group the data matrices 20 generated by the program code from sample segments 17 corresponding to time ranges when the user 32 is walking on stairs and are classified as stable. The B-type stability data group 23 can group the data matrices 20 generated by the program code from sample segments 17 corresponding to time ranges when the user 32 is walking on stairs and are classified as unstable. The activity can be walking, walking on stairs, jogging, running, jumping, dancing, or any other predefined activity.

Optionally, all the classification processes of the second classification process 500 can be structurally similar, but trained with different training data groups, or can differ in at least one hyperparameter, weight values, bias values, and combinations thereof.

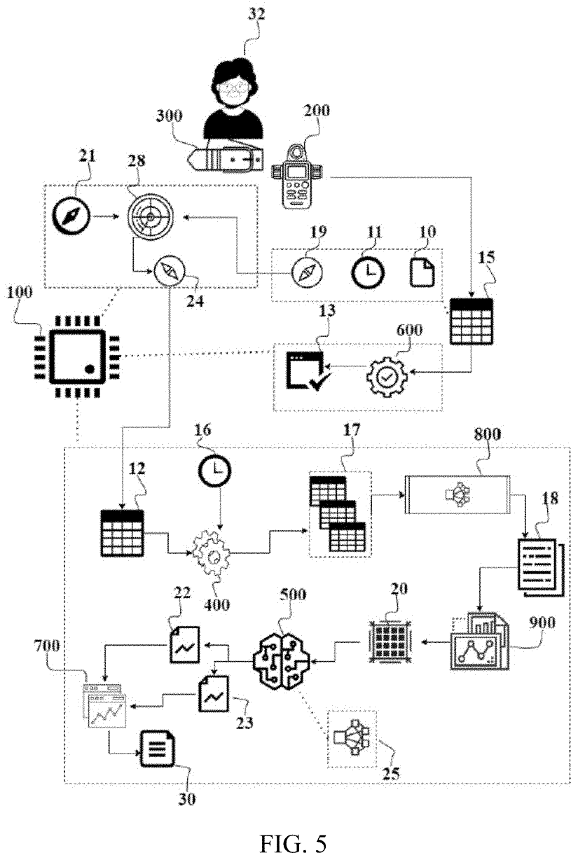

FIG. 5 is a diagram that illustrates various aspects of some embodiments of the present invention. FIG. 5 provides an overview of certain aspects which will be discussed in greater detail herein. As illustrated in FIG. 5, an inertial sensor device 200 is located in a wearable element 300 that is worn by a user 32 and from which a data sample 15 is obtained by the program code (executing on processing resources of the sensor device and/or a computing unit external to the device). The data sample 15 comprises a plurality of data signals 10 and a time vector 11. The program code applied a coordinate axis-shifting process 28 to the data sample 15 to generate a modified data sample 12 to correct for potential changes in the position of the wearable element 300, e.g., when the wearable element 300 deviates from a predetermined position. The program code can apply a preliminary validation process 600 to the data sample 15 to verify that the data is valid data 13. The program code can send the modified data sample 12 to the computing unit 100, which can execute a segmentation process 400 to generate a plurality of sample segments 17 from the valid data 13 for a predetermined time range 16. The computing unit 100 can further execute a first classification process 800 to obtain a gait data group 18 from the plurality of sample segments 17. The computing unit 100 can further execute a wavelet transform process 900 to generate a plurality of data matrices 20 from the gait data group 18. The computing unit 100 can further execute a second classification process 500 to generate data groups 22, 23, for example, A-type stability data group 22 and B-type stability data group 23. The computing unit 100 can further execute a weighting process 700 to generate a stability score data 30 from the data groups 22, 23.

The coordinate axis-shifting process 28 modifies the inertial referral system of a vectorial signal, e.g., acceleration, turning-rate, velocity, etc. The referral system is a vectorial space, for example, the cartesian referral system, polar referral system, spherical referral system, and others in 2D or 3D that are known by a person of ordinary skill in the art. In some examples, the wearable element 300 can shift from a predetermined position and this shift can be detected during the preliminary validation process 600. The coordinate axis-shifting process 28 is configured to mathematically shift the referral system to correct for the adjustment of the wearable element 300.

For example, the coordinate axis-shifting process 28 can be configured to obtain a modified direction vector 24 by shifting a direction vector 19 of the data sample 15, e.g., a direction vector 19 of a vertical acceleration signal contained in the data signals 10 of the data sample 15. A direction vector 19 is the direction along which a signal of the data signals 10 acts. The program code generates a modified direction vector 24 after applying the coordinate axis-shifting process 28. A predetermined coordinate vector 21 is pre-programmed or pre-set for the referral system, each of which has a direction vector to correlate the information in 3D space. A modified data sample 12 results from the application of the coordinate axis-shifting process 28 to the data sample 15.

Referring to FIG. 5, the coordinate axis-shifting process 28 can include program code executing on one or more processors extracting from the data sample 15 a direction vector 19; comparing the direction vector 19 with a predetermined coordinate vector 21; and generating a modified direction vector 24 by shifting the direction vector 19 if the direction vector 19 and the predetermined coordinate vector 21 have a different referral system. If the modified direction vector 24 is generated, the sample segments 17 are generated from a modified data sample 12 that includes said modified direction vector 24. If the direction vector 19 and the predetermined coordinate vector 21 have the same referral system, then a plurality of sample segments 17 can be segmented from the data sample 15 without generating a modified data sample 12.

In some examples, the wearable element 300 can be worn such that the inertial sensor device 200 is in the predetermined location but can also be worn such that the inertial sensor device 200 is in a different position, e.g., upside down or shifted to the side. In such examples, the data sample 15 can still be used if the preliminary validation process 600 detects that the inertial sensor device 200 is shifted from the predetermined location and the coordinate axis-shifting process 28 generates a modified data sample 12 which corrects the data such that it corresponds to data that would have been obtained had the inertial sensor device 200 been positioned in the predetermined location.

FIG. 6 is a diagram that illustrates an aspect of some embodiments of the present invention. As illustrated in FIG. 6, the wavelet transform process 900 is applied to the data signals 10 of the sample segments 17 in a data group (e.g., gait data group 18) to generate data matrices 20. In some examples, the data signals 10 in the sample segments 17 of the data group is convoluted with a mother wave, also known as a wavelet or a scaled wavelet. In some examples, the convolution is a continuous wavelet transformation (CWT). In some examples, the mother wave is a Morlet mother wave. Through the wavelet transform process 900, the data matrix 20 is interpreted as an image, or a scalogram, when plotted and viewed in a user interface, i.e., the time and frequency signals are represented as images, which can be classified by an image classifier. The wavelet transform process 900 can comprise methods and models included in wavelet libraries, such as, but not limited to, Pywavelets (SciPy, PyCWT, ObsPy)), other wavelet libraries known by a person of ordinary skill in the art, and combinations thereof.

FIG. 7 is a diagram that illustrates various aspects of some embodiments of the present invention. FIG. 7 provides an overview of certain aspects which will be discussed in greater detail herein. As illustrated in FIG. 7, data matrices 20 are generated by the program code from the application of the wavelet transform process 900 to data groups 18, e.g., A-type gait data group 33 and B-type gait data group 34. In some examples, A-type gait data group 33 can comprise sample segments 17 corresponding to time ranges when the user 32 is performing A-type gait related activities and B-type gait data groups 34 can comprise sample segments 17 corresponding to time ranges when the user 32 is performing B-type gait related activities.

In some examples, the second classification process 500 can comprise an image classifier, such as an Artificial Neural Networks (ANN), Convolutional Neural Networks (CNN), Autoencoder Neural Networks (AE), Deep Convolutional Networks (DCN), other image classifiers that are known by a person of ordinary skill in the art, and combinations thereof.

In some examples, the convolutional neural network process 25 is a pre-trained CNN configured to classify images. In some examples, the CNN can be MobileNet or MobileNet2 and available in libraries such as Keras, TensorFlow, and other libraries known by a person of ordinary skill in the art. In some examples, the convolutional neural network process 25 can be modified and/or customized to fit the technical requirements of the process disclosed herein, e.g., classifying the data matrices 20 as stability data groups such as A-type stability data group 22 and B-type stability data group 23. For example, the convolutional neural network process 25 can be generated from a pre-trained CNN deleting, modifying, or replacing at least a layer of neurons (e.g., input layer, output layer). The convolutional neural network process 25 can be trained with a training data group configured to allow the convolutional neural network process 25 to classify the A-type stability data group 22, the B-type stability data group 23, both of them, and/or other types of stability data groups 22.

Referring to FIG. 7, in some embodiments, where the convolutional neural network process 25 classifies one or more data groups, the second classification process 500 also comprises one or more neural networks. For example, if the convolutional neural network process 25 classifies an A-type stability data group 22 and a B-type stability data group 22, then the second classification process 500 comprises an A-type neural network 26 and a B-type neural network 27, wherein each type corresponds to a particular user activity 40. Each neural network 26, 27 can assign a score data 39 to each processed sample segment 17, wherein the score data 39 has a value selected from stable and unstable. Further, each neural network 26, 27 can be configured to assign intermediate values of stability, such as a categorical or numerical value according to a predetermined hierarchy or scoring scale, to each sample segment 17 that is processed by the convolutional neural network process 25.

Referring to FIG. 7, the stability score data 30 is generated by the program code through a weighting process 700, which is applied to the A-type stability data group 22 and a B-type stability data group 23 generated by the program code from the second classification process 500.

FIG. 8 is a diagram that illustrates various aspects of some embodiments of the present invention. FIG. 8 provides an overview of certain aspects which will be discussed in greater detail herein. As illustrated in FIG. 8, in one embodiment the system comprises an inertial sensor device 200, a computing device 38, and a server 29, including a computing unit 100. In one embodiment, a data sample 15 is obtained from the inertial sensor device 200 and sent to the computing device 38 through an application, e.g., including API 35, which sends a request data 36 to the server 29, and the server 29 sends a response data 37, along with a stability score data 30, to the computing device 38.

Referring to FIG. 8, the inertial sensor device 200 is located in a wearable element 300 that is worn by a user 32. The wearable element 300 can be configured to position the inertial sensor device 200 at the lower back of the user 32. In some examples, the wearable element 300 can be a clothing element, e.g., a belt, a strap, pants, a shirt, a vest, a jumper, or any other clothing that allows placing the inertial sensor device 200 at the lower back of the user 32. The inertial sensor device 200 can be a hardware element that comprises one or more sensor elements configured to obtain data, including one or more data signals 10, from the user 32. In some examples, the sensor is an accelerometer, gyroscope, magnetometer, equivalent technologies that are known by a person of ordinary skill in the art, or combinations thereof.

Referring to FIG. 8, the inertial sensor device 200 can communicate with the computing device 38, which can be a hardware element configured to establish a connection between the inertial sensor device 200 and a computing unit 100 located in the server 29. In some examples, the computing device 38 is a smartphone, cellphone, tablet, personal computer, smartwatch, microprocessor equipped with internet communication modules and user interfaces, any hardware element that has a processing unit configured to receive instructions from the user 32 or an operator through a human interface device (HID) and a display device configured to display messages and information to the user 32, equivalent technologies that are known by a person of ordinary skill in the art, or combinations thereof. In some examples, the computing device 38 can include a memory module, a wired communications module, a wireless communications module, and other modules that allow recording, consulting, obtaining, sending, and receiving data, commands, requests, and labels. In some examples, a module is implemented as any type of module or component in hardware, software (e.g., instructions that are executable with a processing system), equivalents of such that are known by a person of ordinary skill in the art, or combinations thereof, as a standalone application or as a module or component of another device application, computing device, equivalents that are known by a person of ordinary skill in the art, or combinations thereof.

In some examples, the memory module, or memory register, is a hardware element selected from RAM memories, Flash memories, magnetic hard drives (HDD), solid state disks (SSD), removable memories such as any optical storage, magnetic cassettes, magnetic tapes, or any other memory module that can be used to store information and can be accessed by the computing unit 100, processing unit (e.g., the computing unit 100, the computing device 38), processing module, etc. In some examples, incorporated into the memory module are instructions, data structures, such as text sheets, spreadsheets, or databases, computer program modules, etc. In some examples, a communications module can be a hardware element coupled to the computing unit 100, computing device 38, processing unit, or processing module, which allows establishing communication between one or more computing devices 38, or servers 29, to exchange data, commands, and/or labels, etc. In some examples, the communication module can be a wired communication module comprising a wired connection port allowing for communication with external devices through a communication bus, which can be an inter-integrated circuit (I2C), ethernet, serial peripheral interface (SPI), universal asynchronous receiver transmitter (UART), equivalent technologies that are known by a person of ordinary skill in the art, or combinations thereof. In some examples, the communication module can be a wireless communication module comprising a wireless communication technology, which can be Bluetooth, WiFi, Zigbee, equivalent technologies that are known by a person of ordinary skill in the art, or combinations thereof.

In one embodiment, the computing device 38 is configured to execute and/or access an application 35 that the user 32 interacts with and is in communication with the inertial sensor device 200. In some examples, the APP 35 can be a computer program or method configured to be executed or accessed by the computing device 38 and to allow the computing device 38 to interchange data with the server 29, the user 32, and the inertial sensor device 200. In some examples, the APP 35 is a Progressive Web App (PWA), native APP, hybrid APP, web page, equivalent applications that are known by a person of ordinary skill in the art, or combinations thereof. In some examples, the computer program can be written in Java, Javascript, Perl, PHP and C++, #C, Python, R-Studio, SQL, Swift, Ruby, Delphi, Visual Basic, D, HTML, HTML5, CSS, other programming languages that are known by a person of ordinary skill in the art, and combinations thereof.

In one embodiment, the inertial sensor device 200 can communicate with the computing device 38 via a wired or a wireless connection. In some examples, the inertial sensor device 200 is connected to the computing device 38 by Bluetooth, WiFi, Radio Frequency Identification (RFID), Long Range (LoRa), ZigBee, equivalent wireless communication technologies that are known by a person of ordinary skill in the art, or combinations thereof. In some examples, the inertial sensor device 200 is connected to the computing device 38 by technologies such as USB, I2C, UART, SPI, equivalent technologies that are known by a person of ordinary skill in the art, and combinations thereof.

In one embodiment, the inertial sensor device 200 can be configured to access an application 35 (i.e., APP) that establishes a connection with the server 29. In some examples, the APP 35 can be configured to receive from the computing device 38 the data sample 15 and transmit a request data 36 from the computing device 38 to the server 29. In some examples, the request data 36 includes the data sample 15 and patient information. The server 29 responds to the request data 36 with response data 37, including the stability score data 39, generated by the computing unit 100. The server 29 is configured to generate the stability score data 30 by executing any of the previously described embodiments of the herein disclosed method. The score data 39 indicates a level or class of stability of instability for each sample segment 17. The stability score data 30 is an index or value that indicates how critical is the stability of a user 32 according to the data signals 10 of the data sample 15.

Referring to FIG. 8, the server 29 can comprise a computing unit 100 and establish communication with the computing device 38 through one or more communication protocols in a computer network to exchange data, requests, or data packets used in one or more steps of the method of the present invention. In some examples, the server 29 comprises a processing unit configured to execute a series of instructions corresponding to stages or steps of methods, routines, or processes. In some examples, the server 29 can install and/or run a computer program that can be written in Java, Javascript, Perl, PHP and C++, #C, Python, R-Studio, SQL, Ruby, Delphi, HTML, HTML5, CSS, other programming languages that are known by a person of ordinary skill in the art, or combinations thereof. In some examples, the server 29 can comprise a communications module that allows connection to other servers or computing devices (e.g., the computing device 38). In some examples, the server 29 can connect with other servers and other computing devices through web services architectures and communicate through communication protocols such as SOAP, HTTP/S, MQTT/S, other communication protocols that are known by a person of ordinary skill in the art, or combinations thereof. In some examples, the server 29 is a physical server or a virtual server with a backup architecture or clustered architecture in which one or more replacement servers are configured to guarantee high availability. In some examples, the server 29 comprises a memory module configured to store instructions that, when executed by the server 29 makes the computing unit 100 execute part or all of one or more stages of any of the methods disclosed herein.

Referring to FIG. 8, the computing unit 100 can be a device that processes data, such as a microcontroller, microprocessor, Digital Signal Controller (DSC), Field Programmable Gate Array (FPGA) System on Chip (SoCs), computers, servers, tablets, cellphones, smartphones, any other computer unit, processing unit or processing module that is known by a person of skill in the art, or combinations thereof. In some examples, the computing unit 100 can be a virtual machine or processing module configured to execute one or more steps of the herein disclosed method.

The computing unit 100 can be configured to receive a data sample 15; execute a segmentation process to generate a plurality of sample segments 17 from the data sample 15; execute a first classification process 800 to determine a user activity related data groups 41 from the sample segments 17; apply a wavelet transform process 900 to the data signals 10 of said sample segments 17 to generate a plurality of data matrices 920 for each sample segment 17; execute a second classification process 500 to assign data groups 22, 23 from the data matrices 20 for each user activity related data group 41; and execute a weighting process 700 to generate a stability score 30.

The computing unit 100 can receive instructions from a computer program, wherein said instructions cause the computing unit 100 to carry out the steps of the different embodiments of the method as disclosed herein. In some examples, the computer program is written in Java, Javascript, Perl, PHP and C++, #C, Python, R-Studio, SQL, Swift, Ruby, Delphi, Visual Basic, D, HTML, HTML5, CSS, other programming languages that are known by a person of ordinary skill in the art, and combinations thereof.

The computer program can be stored in a Memory module, which comprises instructions, that when the program is executed by the computing unit 100, cause the computing unit 100 to carry out the steps of the different embodiments of the method disclosed herein.

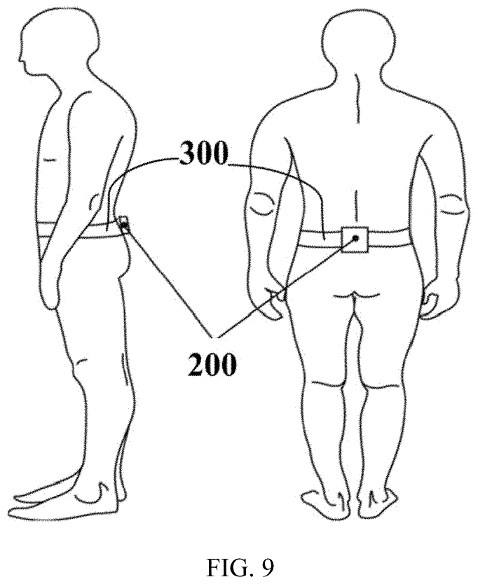

FIG. 9 is a diagram that illustrates various aspects of some embodiments of the present invention. As illustrated in FIG. 9, the inertial sensor device 200, which is located in a wearable element 300, as described in greater detail above.

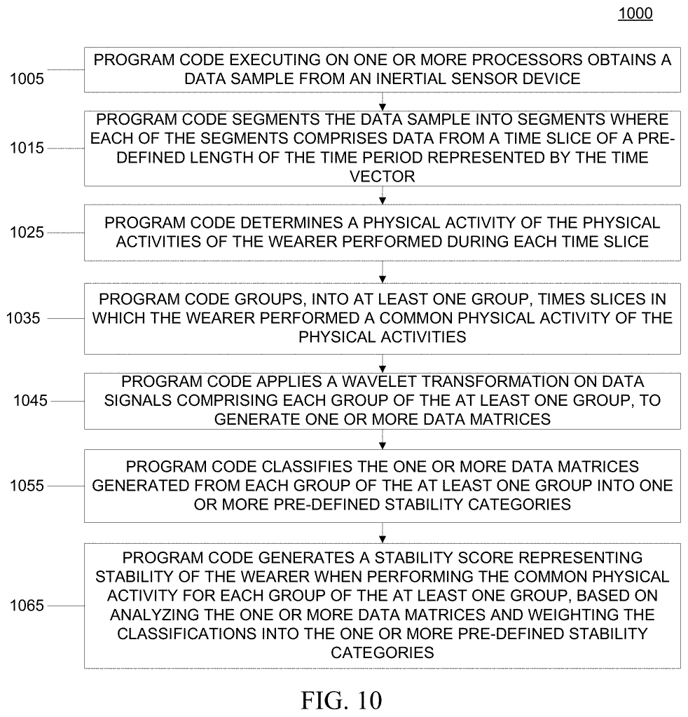

FIG. 10 is a workflow 1000 that illustrates various aspects of some embodiments of the present invention. In some embodiments of the present invention, program code executing on one or more processors obtains a data sample from an inertial sensor device (1005). In this example, the inertial sensor device is a wearable device comprises the inertial sensor device. The data sample is comprised of data signals and a time vector. The program code obtains the data sample from the inertial sensor device based on the inertial sensor device monitoring physical activities of a wearer during a time period represented by the time vector. The program code segments the data sample into segments where each of the segments comprises data from a time slice of a pre-defined length of the time period represented by the time vector (1015).

Referring to FIG. 10, the program code determines a physical activity of the physical activities of the wearer performed during each time slice (1025). In some embodiments, there is a pre-defined set of physical activities in which a given user can be participating that the program code can recognize. For example, training data can be utilized to train the program code to recognize activities from this set of activities based on characteristics of the data obtained by monitoring the wearer. For example, the program code can be trained to recognize that a set of parameters, including acceleration, data, indicates that the wearer is walking. In some examples, the program code can access a database that stores identifying parameters from various activities and can reference data in the database to determine what activity data in a given segments indicates the wearer is performing. The program code groups, into at least one group, times slices in which the wearer performed a common physical activity of the physical activities (1035).

As depicted in FIG. 10, the program code applies a wavelet transformation on data signals comprising each group of the at least one group, to generate one or more data matrices (1045). The program code classifies the one or more data matrices generated from each group of the at least one group into one or more pre-defined stability categories (1055). The program code generates a stability score representing stability of the wearer when performing the common physical activity for each group of the at least one group, based on analyzing the one or more data matrices and weighting the classifications into the one or more pre-defined stability categories (1065).

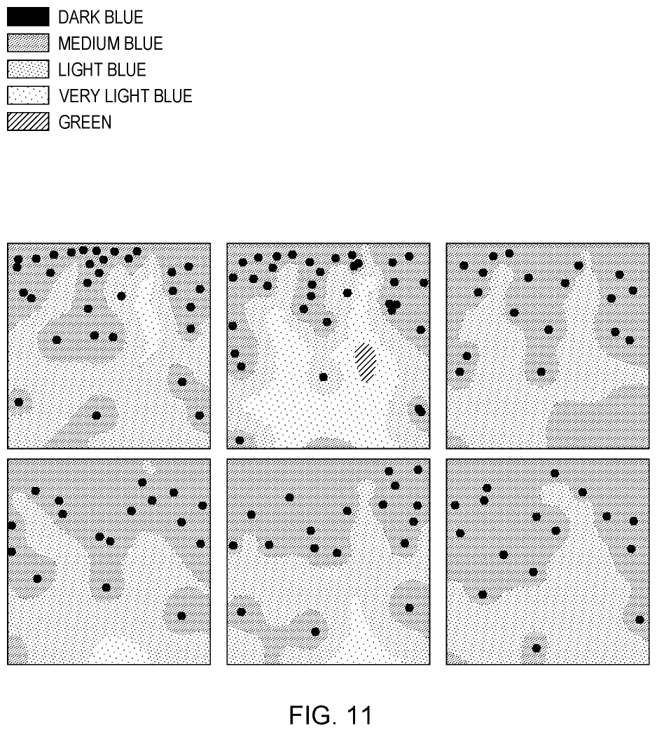

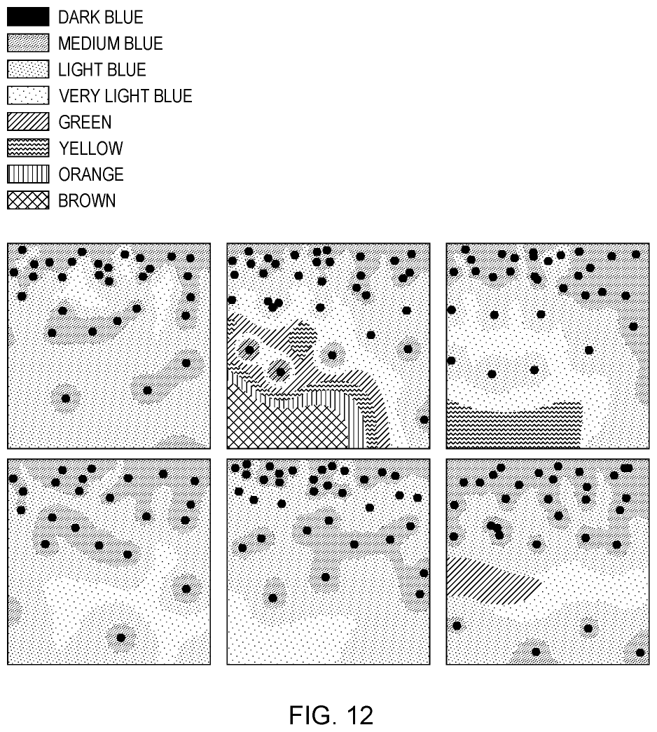

In some examples of the present invention, the wavelet transformation the program code performs on signals comprising each group of the at least one group, to generate one or more data matrices (1045) result in scalograms. It is these scalograms that the program code analyzes in categorizing (1055) and scoring (1065) the stability or lack thereof of an individual (i.e., the wearer of the device). FIGS. 11-12 illustrate scalograms upon which the program code performs the various analyses described herein when the program code determines whether a given individual who is engaged in a given physical activity (e.g., walking, running, etc.) is stable, for that individual within that time period. The illustrations provided in FIGS. 11-12 are extreme visual examples and are provided so that a person looking at these figures can appreciate differences in the illustrations. However, these determinations regarding stability and instability cannot be made by a person, visually, without these hand-selected examples. But these examples are provided here to illustrate aspects that are generally not perceivable by a person. These examples include FIGS. 11-16, which are all discussed in more detail below.

Turning first to FIGS. 11-12, these figures include scalograms of a user engaged in a walking activity, specifically, in walking up stairs. FIG. 11 is a group of scalograms illustrating an individual who is not stable, as determined by the program code, when walking up the stairs, during the defined period of time. FIG. 12 is a group of scalograms illustrating an individual who is stable, as determined by the program code, when walking up the stairs, during the defined period of time. The scalograms in FIGS. 11-12 illustrate the energy frequencies of the movements of the user engaged in the physical activity. As aforementioned, in this example, the physical activity is walking up stairs. An individual who moves more intentionally (i.e., with higher energy frequencies illustrated in the scalograms of FIG. 12) is more stable and an individual who moves less intentionally (i.e., with lower energy frequencies illustrated in the scalograms of FIG. 11), is less stable. There is additional logic in various embodiments of the present invention, including but not limited to where in the time period the various frequency levels are displayed in the scalogram and the shape that the frequency differentials take, given the physical activity, that also indicate the stability or lack thereof of an individual, but as explained earlier, the examples herein are provided as an illustration that can be visually appreciated.

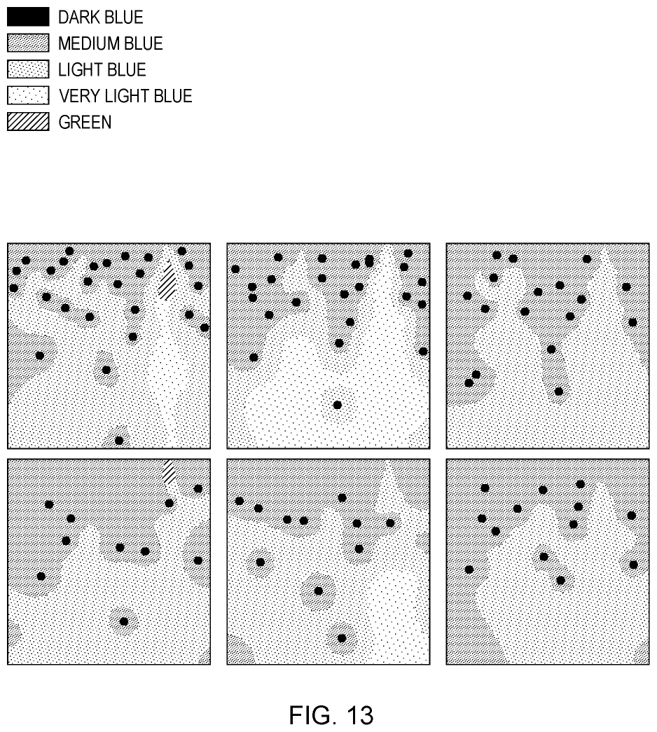

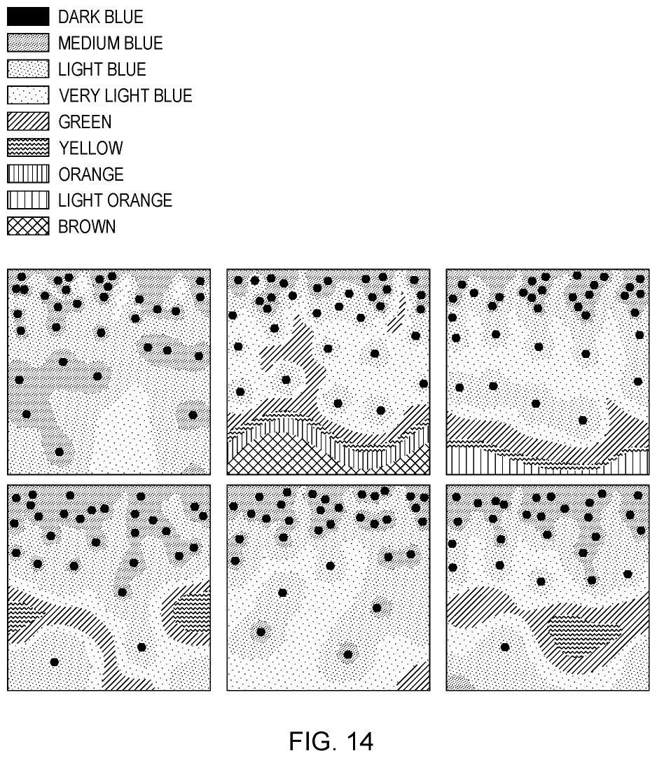

FIGS. 13-14 are also examples of scalograms that contrast an unstable user engaged in walking up to stairs (e.g., FIG. 13), and a stable user engaged in walking up the stairs (e.g., FIG. 14). As is the case in the examples of FIGS. 11-12, FIGS. 13-14 also illustrated how an individual who moves more intentionally (i.e., with higher energy frequencies illustrated in the scalograms of FIG. 14) is more stable and an individual who moves less intentionally (i.e., with lower energy frequencies illustrated in the scalograms of FIG. 13), is less stable. As with the earlier example, these scalograms were chosen specifically because the differences are visible to an individual; these are somewhat extreme examples.

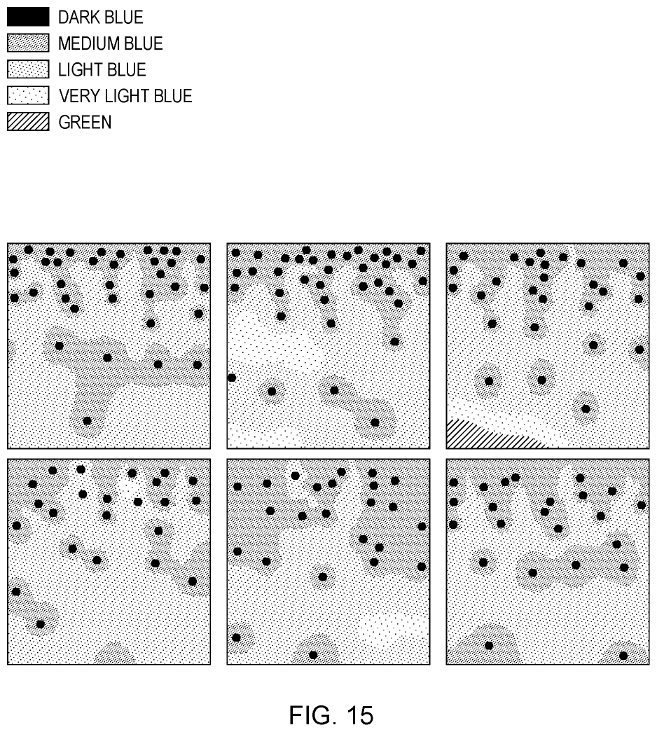

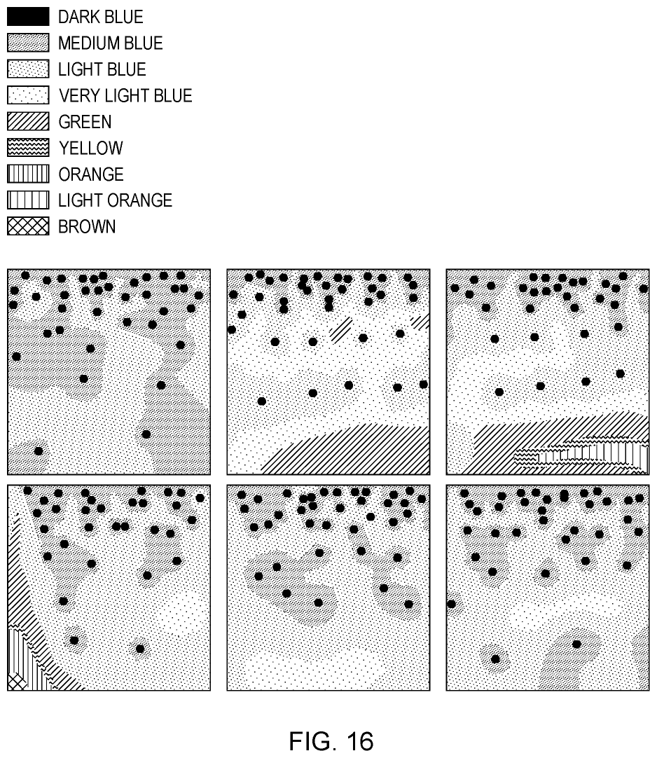

FIGS. 15-16 are examples of scalograms that contrast an unstable user engaged in a walking activity (e.g., FIG. 16), and a stable user engaged in a walking activity (e.g., FIG. 15). As is the case in the examples of FIGS. 11-12, FIGS. 13-14 also illustrated how an individual who moves more intentionally (i.e., with higher energy frequencies illustrated in the scalograms of FIG. 16) is more stable and an individual who moves less intentionally (i.e., with lower energy frequencies illustrated in the scalograms of FIG. 15), is less stable. As with the earlier example, these scalograms were chosen specifically because the differences are visible to an individual; these are somewhat extreme examples.

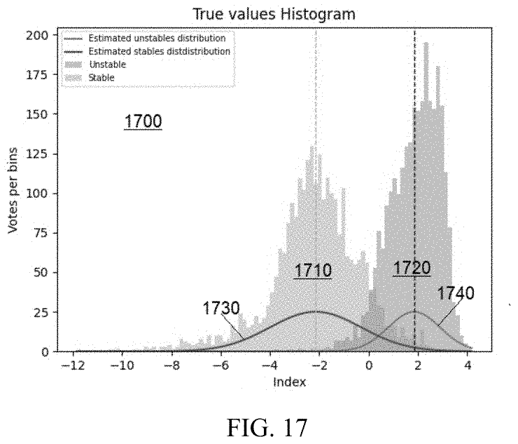

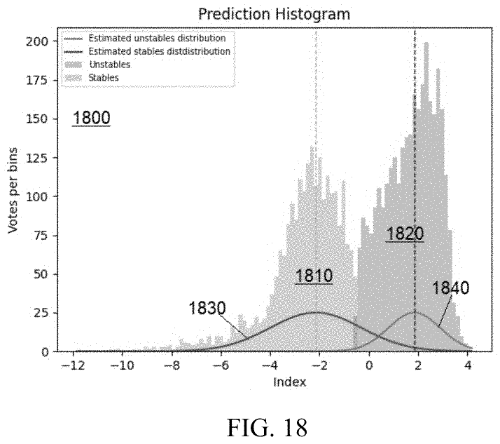

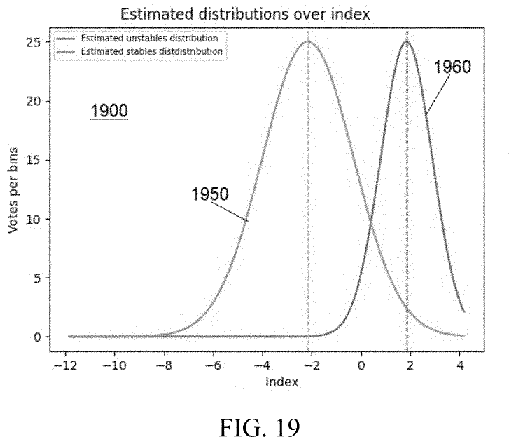

While FIGS. 11-16 include scalograms of an unstable user and a stable user performing walking activities, histograms can be utilized to demonstrates how the program code distributes the segments as stable and unstable and then, how the program code classifies future (time) segments as stable and unstable. FIGS. 17-19 illustrate aspects of how the program code in embodiments of the present invention can evaluate and estimate stability based on evaluating segments from different activities performed by individuals. FIGS. 17-18 are histograms while FIG. 19 is an estimated distribution. FIGS. 17-19 all depict various data and/or calculations by the program code in embodiments of the present invention related to the non-limiting examples of the two dynamic activities of walking and walking on stairs. FIG. 17 is a histogram 1700 that illustrates the labeled segments, including the values indicating stability 1710, and values indicating instability 1720. The program estimates a stable distribution 1730 and an unstable distribution 1740 based on the segments. FIG. 18 is a histogram 1800 that illustrates the program code having applied an algorithm (described in greater detail earlier herein) to the segments. Hence, the program code predicts values indicating stability 1810, and values indicating instability 1820, for a given user or users. The program code also estimates a stable distribution 1830 and an unstable distribution. FIG. 19 illustrates an estimated distribution 1900 for labeled segments, including an estimated distribution for stable segments 1950 and an estimated distribution for unstable segments 1960.

Following are non-limiting examples of various embodiments and different aspects of the present invention. These examples are provided for illustrative purposes only and not to impose any limitations on the Example 1

In a first example of the method, the computing unit 100 receives from the inertial sensor device 200 a data sample 15 having six data signals 10 and a time vector 11 with a magnitude of 60s. The data signals 10 are X-axis acceleration, Y-axis acceleration (measured positive when is parallel to gravity), Z-axis acceleration, X-axis turning rate, Y-axis turning rate, and Z-axis turning rate. The acceleration signals are generated by an accelerometer and the turning rate signals are obtained with a gyroscope.

The program code performs a segmentation process 400 for data obtained within a predetermined time range 16 a value of 3 s. The preliminary validation process 600 generates 20 sample segments 17.

A characteristics vector 14 is obtained for each of these sample segments 17, including values of the means and variances of the 6 signals, producing 12 values (segment characteristics). The program code utilizes a characteristics vector 14 as input and performs the first classification process 800, which is a previously trained classifier that determined to which class the sample segments 17 belong.

In this example, the first classification process 800 is a K-NN classifier.

The first classification process 800 generates a walking data group including sample segments 17 corresponding to time ranges when the user 32 is walking, and a walking on stairs data group 33 that includes sample segments 17 corresponding to time ranges when the user 32 is walking on stairs, it also generates a standing data group including sample segments 17 corresponding to time ranges when the user 32 is standing.

The walking data group includes eight sample segments 17, the walking on stairs data group includes two sample segments 17, and the standing data group includes six sample segments 17. Four sample segments 17 are dropped because they cannot be certainly classified as walking, walking on stairs or standing.

Accordingly, sixteen of the twenty initial sample segments 17 continue to the next step of the method.

After generating the gait data group 18, the computing unit 100 executed the wavelet-transform process 900 and generates the data matrices 20. The wavelet-transform process 900 applies a Continuous Wavelet Transform that takes as mother wavelet a complex morlet wave.

After generating the data matrices 20, the method of the present example executes the second classification process 500 to identify the A-type stability data group 22 and the B-type stability data group 23 for each user activity related data group 41. The A-type stability data group 22 contains the sample segments 17 classified as stable. The B-type stability data group 23 contains the sample segments 17 classified as unstable. In this example, stability is classified in categorical values. In this case the second classification process 500 is a previously trained convolutional neural network process 25 having a previously trained A-type neural network, a previously trained B-type neural network and a previously trained C-type neural network. The A-type neural network is configured to classify the sample segments 17 where the user is walking, the B-type neural network is to classify the sample segments 17 where the user is walking on stairs and the C-type neural network is to classify the sample segments 17 where the user is standing.

In this example, the A-type neural network, the B-type neural network and the C-type neural network are modified CNNs based on a MobileNet-V2 model that is pre-trained on a general and public image database, whose last layers are removed and replaced by fully connected layers (could be a single layer) and then trained specifically to classify the sample segments 17 taking as input the data matrices 20.

Accordingly, the method of this example processes sixteen sample segments 17. Eight sample segments 17 correspond to the walking activities of the user 32, and from said eight sample segments 17, five are classified as stable and three as unstable. Similarly, six sample segments 17 correspond to standing activities of the user 32, from said six sample segments 17, two sample segments 17 are stable, and four sample segment 17 are unstable. Finally, from the two sample segments 17 corresponding to walking on stairs activities, one is stable, and the other is unstable.

Hence, from the sixteen processed sample segments 17 eight are unstable.

In the next step, the method executes the weighting process 700. The weighting process 700 calculates the value for the stability score data 30 according to the following expression:

Stabilityscoredata(30)=(S_{I}/S_{T})*coefₚ+(W_{S}_{_{I}}/W S_{T})*coef_{E}+(W_{I}/W_{T})*coef_{c}

wherein:
W_{I} is the number of sample segments 17 where the user 32 is walking but is unstable.
W_{T} is the number of total sample segments 17 where the user 32 is walking;
W_{S}_{_{I}} is the number of segments 17 where the user 32 is walking on stairs but is unstable;
W_{S}_{_{T}}: is the number of total sample segments 17 where the user 32 is walking; on stairs.
S_{I}: is the number of sample segments 17 where the user 32 is standing but is unstable);
S_{T} is the number of total sample segments 17 where the user 32 is standing. coef_{P}, coef_{E}, coef_{C} are ⅓ each.

In this example the stability score data 30 is a stability score, the higher the value, the more unstable the user 32 is.

The (program code) generated stability score data 30 in this example is 23.6%.

The stability score data 30 can be also expressed categorically, for example, according to a hierarchy pointing if the numerical value of the stability score data 30 is less than 25% the categorical value of the stability score data 30 would be “stable,” if the numerical value of the stability score data 30 is between 25% and 40% the categorical value of the stability score data 30 would be “semi-stable,” or “having a certain grade of stability,” and if the numerical value of the stability score data 30 is more than 40% the categorical value of the stability score data 30 would be “unstable.”

Accordingly, in the present example, the user 32 has a stability score data 30 with a numerical value of 23.6% and a categorical value of “stable.”

### Example 2

In a second example of the method, it further includes a coordinate axis-shifting process 28 where the program code adjust/corrects a direction vector 19 of the data sample 15 obtained by an inertial sensor device 200 located in a wearable element 300 that is worn upside down by the user 32. The coordinate axis-shifting process 28 changes the sign of the direction vector 19 and generates the modified direction vector 24. Also, the coordinate axis-shifting process 28 generates the modified data sample 12, which is employed in the steps of example 1 instead of the data sample 15.

### Example 3

The method of examples 1 and 2 are executed by a system including a computing unit 100 that is located in a remote server 29, such as a web server. The computing device 38 is a smartphone. The APP 35 is a Progressive Web App (PWA) application that establishes with the server 29 a service-based communications protocol, in this case, API-REST.

The inertial sensor device 200 is a Shimmer3 IMU Unit. The inertial sensor device 200 includes a gyroscope and an accelerometer. The wearable element 300 is a waist strap. The inertial sensor device 200 communicates with the computing device 38 by Bluetooth. The computing device 38 executes a data-screening process that enables the program code to obtain the data samples 15. The inertial sensor device 200 transmits to the computing device 38 a CSV file including the six signals (X-acceleration, Y-acceleration, Z-acceleration, X-turning-rate, Y-turning-rate, Z-turning-rate).

The computing device 38 generates a request data 36 that is sent in the APP 35. The request data 36 includes the CSV file that contains the data sample 15. The request data 36 includes identification data configured to identify the user 32 and/or the computing device 38 from with the request data 36 is sent. The server 29 receives the request data 36 and processes the data sample 15 according to the method of Example 1. The server 29 can process the data sample 15 with any of the embodiments of the method herein disclosed. The server 29, with its computing unit 100, executing program code, generates the stability score data 30 and generates a response data 37 that includes the stability score data 30. The server 29 sends the response data 37 to the computing device 38 by the APP 35. After receiving the response data 37, the computing device 38 deploys a message in a user interface (e.g., the screen of the smartphone) displaying the stability score data 30 results (numerical and/or categorical values).

### Example 4

In this example, the method is the same as in the Example 1 but for the A-type neural network and the B-type neural network that are trained using autoencoders taking as input the data matrices 20. The autoencoder is trained to reconstruct the data matrices 20. The autoencoder dense layer output is used to train a LDA classifier, which classifies the sample segment 17 into stables and unstables.

The “Encoder” stage can be composed with 2D convolutional layers, with “elu” activation, zero padding “same”, and convolutional kernel size “3×3”, and 2D max polling with size “2×2” and zero padding “same”. The “Latent” layer is a flattened dense layer, with size “256”. The “Decoding” stage is inverse to the “Encoding” stage.

In this example of the method, the passive-activity data group is taken as input for a C-classification process comprising a model that considers the user 32 as an inverted-pendulum, that also classifies the sample segments 17 into stables and unstable.

Embodiments of the present invention include a computer-implemented method, a computer-program product, and a computer system where one or more processors performs a method. The method can include the processor(s) obtaining a data sample from an inertial sensor device, where a wearable device comprises the inertial sensor device, where the data sample is comprised of data signals and a time vector, where the data sample is obtained by the inertial sensor device based on the inertial sensor device monitoring physical activities of a wearer during a time period represented by the time vector; segmenting the data sample into segments, wherein each segment comprises data from a time slice of the time period represented by the time vector; determining a physical activity of the physical activities of the wearer performed during each time slice; grouping, by, into at least one group, times slices in which the wearer performed a common physical activity of the physical activities; applying a change of basis transformation on data signals comprising each group of the at least one group, to generate one or more data matrices; classifying the one or more data matrices generated from each group of the at least one group into one or more pre-defined stability categories; and generating a stability score representing stability of the wearer when performing the common physical activity for each group of the at least one group, based on analyzing the one or more data matrices and weighting the classifications into the one or more pre-defined stability categories.

In some examples, the change of basis transformation comprises a wavelet transformation.

In some examples, each time slice comprises: a pre-defined length of time or a quantifiable number of acts completed in a given physical activity of the physical activities during the time slice.

In some examples, determining the physical activity is based on one or more of: a portion of the data sample from the inertial sensor device, or data entry by the wearer through an interface of the inertial sensor device.

In some examples, the processor(s) automatically alerts at least one pre-configured contact, of the stability score, via a communications network.

In some examples, a given group of the at least one group comprises a gait group.

In some examples, the pre-defined stability categories comprise a first category and a second category, wherein the first category indicates stability and the second category indicates instability.

In some examples, the inertial sensor device comprises one inertial measurement unit sensor.

In some examples, prior to the segmenting, the processor(s) validates the data sample, where the validating comprises: determining if the data signals comprising the data sample indicate that the wearer was in motion during the time period; and based on determining, that the wearer was in motion during the time period, commencing the segmenting.

In some examples, prior to the segmenting, the processor(s) validates the data sample, where the validating comprises: determining if the data signals comprising the data sample indicate that the wearer was in motion during the time period; and based on determining that the wearer was in not motion during the time period, obtaining, from the inertial sensor device, additional data over a new period of time, wherein based on the determining, the new period of time comprises the time period and the additional data comprises the data sample.

In some examples, based on based determining that the wearer was in not motion during the time period, the processor(s) alerts the wearer that the inertial sensor device will collect additional data.

In some examples, the one or more data matrices comprise scalograms.

In some examples, the data signals are selected from the group consisting of: three-axis-low noise acceleration, three-axis-wide range acceleration, and three-axis-gyroscopic angular rate.

In some examples, the data signals comprise acceleration data and determining if the data signals comprising the data sample indicate that the wearer was in motion during the time period comprises the processor(s) determining that the acceleration data is within a predetermined acceleration range.

In some examples, the processor(s) obtaining the data sample comprises: executing coordinate axis-shifting, the coordinate shifting comprising: extracting a direction vector from the data sample; determining if the direction vector and a predetermined coordinate vector have a different referral system, based on comparing the direction vector to the predetermined coordinate vector; based on determining that the direction vector and the predetermined coordinate vector have the different referral system, generating a modified direction vector to replace the direction vector; and augmenting the data sample to include the modified direction vector in place of the direction vector.

In some examples, the physical activity of the physical activities of the wearer performed during each time slice comprises: identifying the physical activity, based on at least one parameter of data comprising each segment selected from the group consisting of: mean, variance, standard deviation, energy, leading frequencies, maximum values, minimum values, and correlation values.

In some examples, the processor(s) classifying the one or more data matrices into one or more pre-defined stability categories comprises: training with training data, a convolutional neural network process to classify segments into a finite number of groups, wherein each group represents a distinct physical activity of the physical activities; and utilizing, the previously trained convolutional neural network process, to classify the one or more data matrices into the one or more pre-defined stability categories.

In some examples, each group of the finite number of groups represents a distinct physical activity selected from the group consisting of: walking group, walking on stairs, and standing.

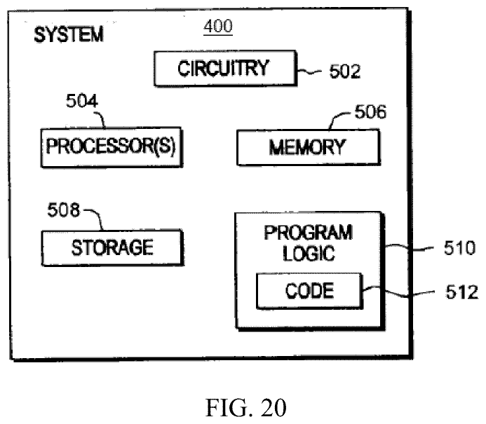

FIG. 20 illustrates a block diagram of a resource 400 in computer system, such as, which is part of the technical architecture of certain embodiments of the technique. For example, a resource 400 could be connected to or included in the modems utilized in various embodiments of the present invention to send and receive the additional signal over the legacy bus. Additionally, certain buses that can be utilized in embodiments of the present invention are themselves computing resources 400. Returning to FIG. 20, the resource 400 may include a circuitry 502 that may in certain embodiments include a microprocessor 504. The computer system 400 may also include a memory 506 (e.g., a volatile memory device), and storage 508. The storage 508 may include a non-volatile memory device (e.g., EEPROM, ROM, PROM, RAM, DRAM, SRAM, flash, firmware, programmable logic, etc.), magnetic disk drive, optical disk drive, tape drive, etc. The storage 508 may comprise an internal storage device, an attached storage device and/or a network accessible storage device. The system 400 may include a program logic 510 including code 512 that may be loaded into the memory 506 and executed by the microprocessor 504 or circuitry 502.

In certain embodiments, the program logic 510 including code 512 may be stored in the storage 508, or memory 506. In certain other embodiments, the program logic 510 may be implemented in the circuitry 502. Therefore, while FIG. 7 shows the program logic 510 separately from the other elements, the program logic 510 may be implemented in the memory 506 and/or the circuitry 502. The program logic 510 may include the program code discussed in this disclosure that facilitates the reconfiguration of elements of various computer networks, including those in various figures.

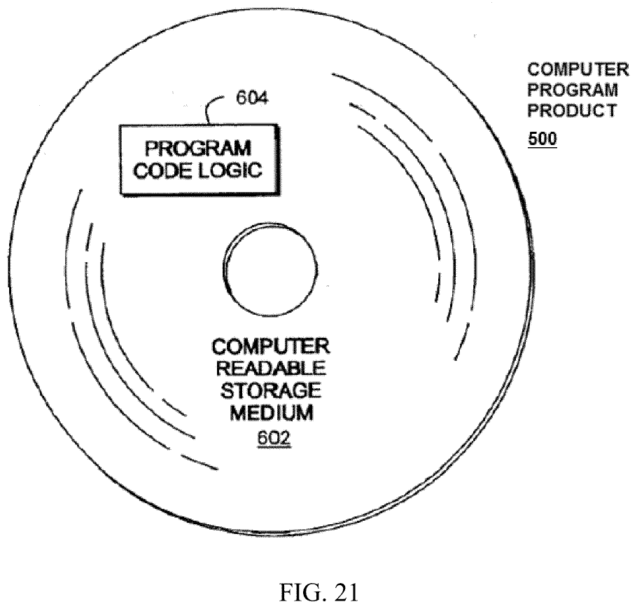

Using the processing resources of a resource 400 to execute software, computer-readable code or instructions, does not limit where this code can be stored. Referring to FIG. 21, in one example, a computer program product 500 includes, for instance, one or more non-transitory computer readable storage media 602 to store computer readable program code means or logic 604 thereon to provide and facilitate one or more aspects of the technique.

As will be appreciated by one skilled in the art, aspects of the technique may be embodied as a system, method or computer program product. Accordingly, aspects of the technique may take the form of an entirely hardware embodiment, an entirely software embodiment (including firmware, resident software, micro-code, etc.) or an embodiment combining software and hardware aspects that may all generally be referred to herein as a “circuit,” “module” or “system”. Furthermore, aspects of the technique may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable signal medium may include a propagated data signal with computer readable program code embodied therein, for example, in baseband or as part of a carrier wave. Such a propagated signal may take any of a variety of forms, including, but not limited to, electro-magnetic, optical or any suitable combination thereof. A computer readable signal medium may be any computer readable medium that is not a computer readable storage medium and that can communicate, propagate, or transport a program for use by or in connection with an instruction execution system, apparatus, or device.

A computer readable storage medium may be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Any combination of one or more computer readable medium(s) may be utilized. The computer readable medium may be a computer readable signal medium or a computer readable storage medium. A computer readable signal medium may include a propagated data signal with computer readable program code embodied therein, for example, in baseband or as part of a carrier wave. Such a propagated signal may take any of a variety of forms, including, but not limited to, electro-magnetic, optical or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using an appropriate medium, including but not limited to wireless, wireline, optical fiber cable, RF, etc., or any suitable combination of the foregoing.

Computer program code for carrying out operations for aspects of the technique may be written in any combination of one or more programming languages, including an object oriented programming language, such as Java, Smalltalk, C++ or the like, and conventional procedural programming languages, such as the “C” programming language, PHP, ASP, assembler or similar programming languages, as well as functional programming languages and languages for technical computing (e.g., Python, Matlab). The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). Furthermore, more than one computer can be used for implementing the program code, including, but not limited to, one or more resources in a cloud computing environment.

Aspects of the technique are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions, also referred to as software and/or program code, may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the technique. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In addition to the above, one or more aspects of the technique may be provided, offered, deployed, managed, serviced, etc. by a service provider who offers management of customer environments. For instance, the service provider can create, maintain, support, etc. computer code and/or a computer infrastructure that performs one or more aspects of the technique for one or more customers. In return, the service provider may receive payment from the customer under a subscription and/or fee agreement, as examples. Additionally or alternatively, the service provider may receive payment from the sale of advertising content to one or more third parties.

In one aspect of the technique, an application may be deployed for performing one or more aspects of the technique. As one example, the deploying of an application comprises providing computer infrastructure operable to perform one or more aspects of the technique.

As a further aspect of the technique, a computing infrastructure may be deployed comprising integrating computer readable code into a computing system, in which the code in combination with the computing system is capable of performing one or more aspects of the technique.

As yet a further aspect of the technique, a process for integrating computing infrastructure comprising integrating computer readable code into a computer system may be provided. The computer system comprises a computer readable medium, in which the computer medium comprises one or more aspects of the technique. The code in combination with the computer system is capable of performing one or more aspects of the technique.

Further, other types of computing environments can benefit from one or more aspects of the technique. As an example, an environment may include an emulator (e.g., software or other emulation mechanisms), in which a particular architecture (including, for instance, instruction execution, architected functions, such as address translation, and architected registers) or a subset thereof is emulated (e.g., on a native computer system having a processor and memory). In such an environment, one or more emulation functions of the emulator can implement one or more aspects of the technique, even though a computer executing the emulator may have a different architecture than the capabilities being emulated. As one example, in emulation mode, the specific instruction or operation being emulated is decoded, and an appropriate emulation function is built to implement the individual instruction or operation.

In an emulation environment, a host computer includes, for instance, a memory to store instructions and data; an instruction fetch unit to fetch instructions from memory and to optionally, provide local buffering for the fetched instruction; an instruction decode unit to receive the fetched instructions and to determine the type of instructions that have been fetched; and an instruction execution unit to execute the instructions. Execution may include loading data into a register from memory; storing data back to memory from a register; or performing some type of arithmetic or logical operation, as determined by the decode unit. In one example, each unit is implemented in software. For instance, the operations being performed by the units are implemented as one or more subroutines within emulator software.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Input/Output or I/O devices (including, but not limited to, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or steps plus function elements in the descriptions below, if any, are intended to include any structure, material, or act for performing the function in combination with other elements as specifically noted. The description of the technique has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular uses contemplated.

What is claimed is:

1. A computer-method for determining a user stability score, the method comprising:
obtaining, by one or more processors, a data sample from an inertial sensor device, wherein a wearable device comprises the inertial sensor device, wherein the data sample is comprised of data signals and a time vector, wherein the data sample is obtained by the inertial sensor device based on the inertial sensor device monitoring physical activities of a wearer during a time period represented by the time vector;
segmenting, by the one or more processors, the data sample into segments, wherein each segment comprises data from a time slice of the time period represented by the time vector;
determining, by the one or more processors, a physical activity of the physical activities of the wearer performed during each time slice;
grouping, by the one or more processors, into at least one group, times slices in which the wearer performed a common physical activity of the physical activities;
applying, by the one or more processors, a change of basis transformation on data signals comprising each group of the at least one group, to generate one or more data matrices;
classifying, by the one or more processors, the one or more data matrices generated from each group of the at least one group into one or more pre-defined stability categories; and
generating, by the one or more processors, a stability score representing stability of the wearer when performing the common physical activity for each group of the at least one group, based on analyzing the one or more data matrices and weighting the classifications into the one or more pre-defined stability categories.

2. The computer-implemented method of claim 1, wherein the change of basis transformation comprises a wavelet transformation.

3. The computer-implemented of claim 1, wherein each time slice comprises: a pre-defined length of time or a quantifiable number of acts completed in a given physical activity of the physical activities during the time slice.

4. The computer-implemented method of claim 1, wherein determining the physical activity is based on one or more of: a portion of the data sample from the inertial sensor device, or data entry by the wearer through an interface of the inertial sensor device.

5. The computer-implemented method of claim 1, further comprising:
automatically alerting, by the one or more processors, at least one pre-configured contact, of the stability score, via a communications network.

6. The computer-implemented method of claim 1, wherein the a given group of the at least one group comprises a gait group.

7. The computer-implemented method of claim 1, wherein the pre-defined stability categories comprise a first category and a second category, wherein the first category indicates stability and the second category indicates instability.

8. The computer-implemented method of claim 1, wherein the inertial sensor device comprises one inertial measurement unit sensor.

9. The computer-implemented method of claim 1, further comprising:
prior to the segmenting, validating, by the one or more processors, the data sample, wherein the validating comprises:
determining, by the one or more processors, if the data signals comprising the data sample indicate that the wearer was in motion during the time period; and
based on determining, by the one or more processors, that the wearer was in motion during the time period, commencing the segmenting.

10. The computer-implemented method of claim 1, further comprising:
prior to the segmenting, validating, by the one or more processors, the data sample, wherein the validating comprises:
determining, by the one or more processors, if the data signals comprising the data sample indicate that the wearer was in motion during the time period; and
based on determining, by the one or more processors, that the wearer was in not motion during the time period, obtaining, from the inertial sensor device, additional data over a new period of time, wherein based on the determining, the new period of time comprises the time period and the additional data comprises the data sample.

11. The computer-implemented method of claim 10, further comprising:
based on based determining that the wearer was in not motion during the time period, alerting, by the one or more processors, the wearer that the inertial sensor device will collect additional data.

12. The computer-implemented method of claim 1, wherein the one or more data matrices comprise scalograms.

13. The computer-implemented method of claim 1, wherein the data signals are selected from the group consisting of: three-axis-low noise acceleration, three-axis-wide range acceleration, and three-axis-gyroscopic angular rate.

14. The computer-implemented method of claim 8, wherein the data signals comprise acceleration data and wherein determining if the data signals comprising the data sample indicate that the wearer was in motion during the time period comprises determining that the acceleration data is within a predetermined acceleration range.

15. The computer-implemented method of claim 1, wherein obtaining the data sample comprises:
executing, by the one or more processors, coordinate axis-shifting, the coordinate shifting comprising:
extracting, by the one or more processors, a direction vector from the data sample;
determining, by the one or more processors, if the direction vector and a predetermined coordinate vector have a different referral system, based on comparing the direction vector to the predetermined coordinate vector;
based on determining, by the one or more processors, that the direction vector and the predetermined coordinate vector have the different referral system, generating a modified direction vector to replace the direction vector; and
augmenting, by the one or more processors, the data sample to include the modified direction vector in place of the direction vector.

16. The computer-implemented method of claim 1, wherein the physical activity of the physical activities of the wearer performed during each time slice comprises:
identifying, by the one or more processors, the physical activity, based on at least one parameter of data comprising each segment selected from the group consisting of: mean, variance, standard deviation, energy, leading frequencies, maximum values, minimum values, and correlation values.

17. The computer-implemented method of claim 1, wherein classifying the one or more data matrices into one or more pre-defined stability categories comprises:
training, by the one or more processors, with training data, a convolutional neural network process to classify segments into a finite number of groups, wherein each group represents a distinct physical activity of the physical activities; and
utilizing, by the one or more processors, the previously trained convolutional neural network process, to classify the one or more data matrices into the one or more pre-defined stability categories.

18. The computer-implemented method of claim 17, wherein each group of the finite number of groups represents a distinct physical activity selected from the group consisting of:
walking group, walking on stairs, and standing.

19. A computer system comprising:
a memory;
one or more processors in communication with the memory;
a sensor device in communication with the one or more processors;
program instructions executable by the one or more processors via the memory to perform a method, the method comprising:
transmitting, by the sensor device, to the one or more processors, a data sample from the sensor device, wherein the data sample is comprised of data signals and a time vector, wherein the data sample is obtained by the sensor device based on the sensor device monitoring physical activities of an individual during a time period represented by the time vector;
segmenting, by the one or more processors, the data sample into segments, wherein each segment comprises data from a time slice of the time period represented by the time vector;
determining, by the one or more processors, a physical activity of the physical activities of the wearer performed during each time slice;
grouping, by the one or more processors, into at least one group, times slices in which the wearer performed a common physical activity of the physical activities;
applying, by the one or more processors, a change of basis transformation on data signals comprising each group of the at least one group, to generate one or more data matrices;
classifying, by the one or more processors, the one or more data matrices generated from each group of the at least one group into one or more pre-defined stability categories; and
generating, by the one or more processors, a stability score representing stability of the wearer when performing the common physical activity for each group of the at least one group, based on analyzing the one or more data matrices and weighting the classifications into the one or more pre-defined stability categories.

20. A computer program product comprising:
a computer readable storage medium readable by one or more processors of a computing system and storing instructions for execution by the one or more processors for performing a method comprising:
obtaining, by the one or more processors, a data sample from an inertial sensor device, wherein a wearable device comprises the inertial sensor device, wherein the data sample is comprised of data signals and a time vector, wherein the data sample is obtained by the inertial sensor device based on the inertial sensor device monitoring physical activities of a wearer during a time period represented by the time vector;
segmenting, by the one or more processors, the data sample into segments, wherein each segment comprises data from a time slice of the time period represented by the time vector;
determining, by the one or more processors, a physical activity of the physical activities of the wearer performed during each time slice;
grouping, by the one or more processors, into at least one group, times slices in which the wearer performed a common physical activity of the physical activities;
applying, by the one or more processors, a change of basis transformation on data signals comprising each group of the at least one group, to generate one or more data matrices;
classifying, by the one or more processors, the one or more data matrices generated from each group of the at least one group into one or more pre-defined stability categories; and
generating, by the one or more processors, a stability score representing stability of the wearer when performing the common physical activity for each group of the at least one group, based on analyzing the one or more data matrices and weighting the classifications into the one or more pre-defined stability categories.
